(19) United States
(12) Patent Application Publication
(10) Pub. No.: US 2005/0014685 A1
(43) Pub. Date: Jan. 20, 2005

(54) New compound

(75) Inventors: Hiroaki Mizuno, Osaka (JP); Hiroshi Matsuda, Osaka (JP); Takahiro Matsuya, Osaka (JP); David Barrett, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: FUJISAWA PHARMACEUTICAL CO., LTD., Osaka-shi (JP)

(21) Appl. No.: 10/873,305

(22) Filed: Jun. 23, 2004

(30) Foreign Application Priority Data
Jul. 14, 2003 (AU) 2003903629

(51) Int. Cl. C07K 7/64; A61K 38/12

(52) U.S. Cl. 514/9; 530/317

(57) ABSTRACT

This invention relates to new lipopeptide compound represented by the following general formula (I): 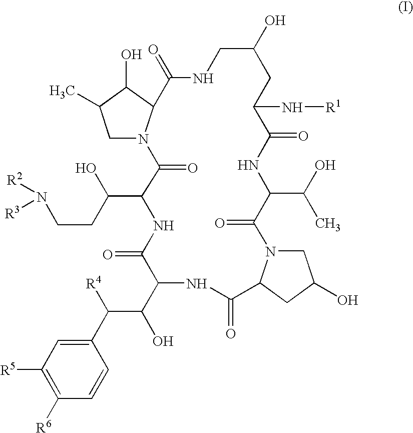 Wherein
R¹, R², R³, R⁴, R⁵ and R⁶ are as defined in the description or a salt thereof which has antimicrobial activities (especially, antifungal activities), inhibitory activity on β-1,3-glucan synthase, to process for preparation thereof, to a pharmaceutical composition comprising the same, and to a method for prophylactic and/or therapeutic treatment of infectious diseases including Pneumocystis carinii infection (e.g. Pneumocystis carinii pneumonia) in a human being or an animal.

### TECHNICAL FIELD

The present invention relates to new lipopeptide compounds and salts thereof which are useful as a medicament.

### BACKGROUND ART

In U.S. Pat. Nos. 5,376,634, 5,569,646, WO 96/11210 and WO 99/40108, WO 00/64927 and WO 01/60846, there are disclosed the lipopeptide compound and a pharmaceutically acceptable salt thereof, which have antimicrobial activities (especially antifungal activity).

### DISCLOSURE OF INVENTION

The present invention relates to new lipopeptide compound and a salt thereof.

More particularly, it relates to new lipopeptide compound and a salt thereof, which have antimicrobial activities [especially, antifungal activities, in which the fungi may include Aspergillus, Cryptococcus, Candida, Mucor, Actinomyces, Histoplasma, Dermatophyte, Malassezia, Fusarium and the like.], inhibitory activity on β-1,3-glucan synthase, and further which are expected to be useful for the prophylactic and/or therapeutic treatment of Pneumocystis carinii infection (e.g. Pneumocystis carinii pneumonia) in a human being or an animal, to a process for preparation thereof, to a pharmaceutical composition comprising the same, and to a method for the prophylactic and/or therapeutic treatment of infectious disease including Pneumocystis carinii infection (e.g. Pneumocystis carinii pneumonia) in a human being or an animal.

The object lipopeptide compounds of the present invention are new and can be represented by the following general formula (I): 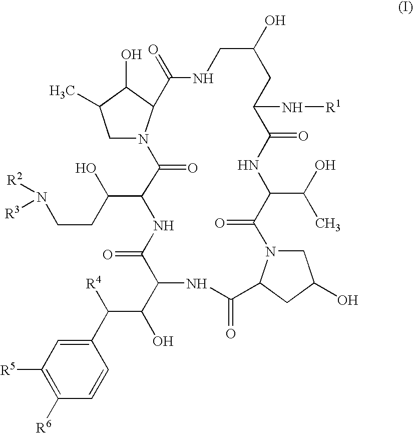
wherein
R¹ is hydrogen or acyl group,
R² is hydrogen or amino protective group,
R³ is hydrogen or lower alkyl substituted with one or more hydroxy,
R⁴ is hydrogen or hydroxy,
R⁵ is lower alkoxy, and
R⁶ is hydroxy or acyloxy, or
or a salt thereof.

The new lipopeptide compound (I) or a salt thereof can be prepared by the process as illustrated in the following reaction schemes. 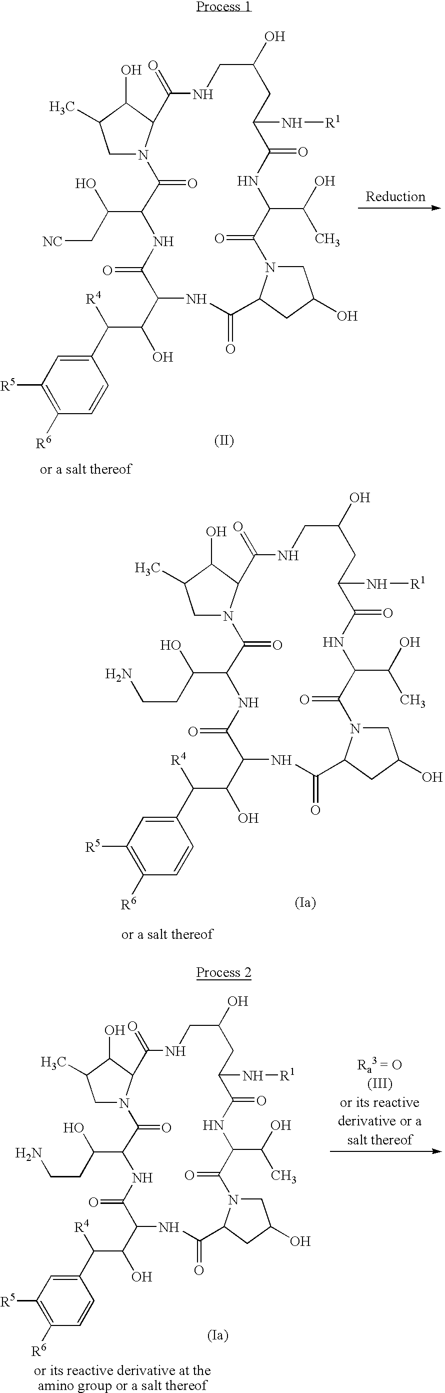 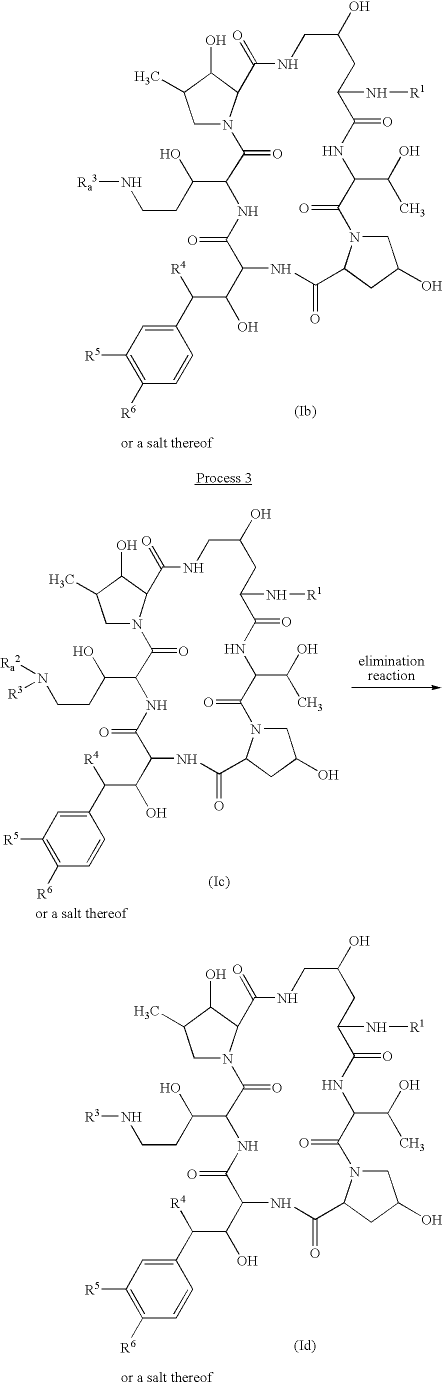 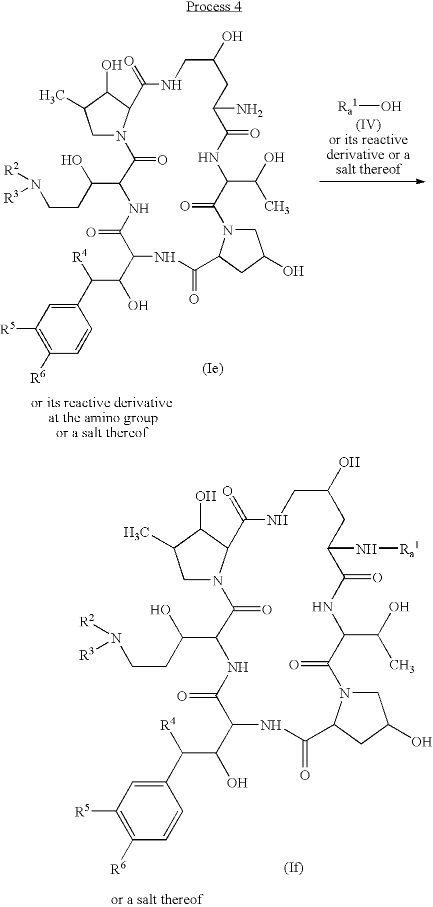
wherein
R¹, R², R³, R⁴, R⁵ and R⁶ are defined above,
Rₐ¹ is acyl group,
Rₐ² is amino protective group, and
Rₐ³ is lower alkyl substituted with one or more hydroxy.

Suitable salt of the new lipopeptide compound (I) is a pharmaceutically acceptable and conventional non-toxic salt, and may include a salt with a base or an acid addition salt such as a salt with an inorganic base, for example, an alkali metal salt (e.g., sodium salt, potassium salt, etc.), an alkaline earth metal salt (e.g., calcium salt, magnesium salt, etc.), an ammonium salt;
a salt with an organic base, for example, an organic amine salt (e.g., triethylamine salt, diisopropylethylamine salt, pyridine salt, picoline salt, ethanolamine salt, triethanolamine salt, dicyclohexylamine salt, N,N′-dibenzylethylenediamine salt, 4-dimethylaminopyridine salt, etc.);
an inorganic acid addition salt (e.g., hydrochloride hydrobromide, sulfate, phosphate, etc.);
an organic carboxylic sulfonic acid addition salt (e.g., formate, acetate, trifluoroacetate, maleate, tartrate, fumarate, methanesulfonate, benzenesulfonate, toluenesulfonate, etc.);
a salt with a basic or acidic amino acid (e.g., arginine, aspartic acid, glutamic acid, etc.).

Suitable examples and illustration of the various definitions in the above and subsequent descriptions of the present specification, which the present invention intends to include within the scope thereof, are explained in detail as follows:

The term “lower” is used to intend a group having 1 to 6 carbon atom(s), unless otherwise provided.

The term “higher” is used to intend a group having 7 to 20 carbon atom(s), and the most preferred one may be the number of 7 to 10, unless otherwise provided.

Suitable example of “one or more” may be the number of 1 to 6, in which the preferred one may be the number of 1 to 3, and the most preferred one may be the number of 1 or 2.

Suitable example of “halogen” may be fluorine, chlorine, bromine, iodine and the like.

Suitable example of “lower alkoxy” may include straight or branched one such as methoxy, ethoxy, propoxy, isopropoxy, butoxy, isobutoxy, tert-butoxy, pentyloxy, tert-pentyloxy, neo-pentyloxy, hexyloxy, isohexyloxy and the like.

Suitable example of “higher alkoxy” may include straight or branched one such as heptyloxy, octyloxy, 3,5-dimethyloctyloxy, 3,7-dimethyloctyloxy, nonyloxy, decyloxy, undecyloxy, dodecyloxy, tridecyloxy, tetradecyloxy, hexadecyloxy, heptadecyloxy, octadecyloxy, nonadecyloxy, icosyloxy, and the like.

Suitable example of “lower alkyl” may include straight or branched one having 1 to 6 carbon atom(s), such as methyl, ethyl, propyl, isopropyl, butyl, isobutyl, sec-butyl, tert-butyl, pentyl, tert-pentyl, neo-pentyl, hexyl, isohexyl, and the like.

Suitable example of “higher alkyl” may include straight or branched one such as heptyl, octyl, 3,5-dimethyloctyl, 3,7-dimethyloctyl, nonyl, decyl, undecyl, dodecyl, tridecyl, tetradecyl, hexadecyl, heptadecyl, octadecyl, nonadecyl, icosyl, and the like.

Suitable example of “lower alkylidene” may include straight or branched one having 1 to 6 carbon atom(s), such as methylidene, ethylidene, propylidene, isopropylidene, and the like.

Suitable example of “aryl” and “ar” moiety may include phenyl which may have lower alkyl (e.g., phenyl, mesityl, xylyl, tolyl, etc.), naphthyl, anthryl, indanyl, fluorenyl, and the like, and this “aryl” and “ar” moiety may have one or more halogen.

Suitable example of “aroyl” may include benzoyl, toluoyl, naphthoyl, anthrylcarbonyl, and the like.

Suitable example of “heterocyclic group” may include
unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g. 1H-tetrazolyl; 2H-tetrazolyl, etc.), etc.;
saturated 3 to 8-membered (more preferably 5 or 6-membered) heteromonocyclic group containing 1 to 4 nitrogen atom(s), for example, pyrrolidinyl, imidazolidinyl, piperidyl, piperazinyl, azetidinyl, etc.;
unsaturated condensed heterocyclic group containing 1 to 4 nitrogen atom(s), for example, indolyl, isoindolyl, indolinyl, indolizinyl, benzimidazolyl, quinolyl, isoquinolyl, indazolyl, benzotriazolyl, etc.;
unsaturated 3 to 8-membered (more preferably 5 or 6-membered) heteromonocyclic group containing 1 or 2 oxygen atom(s) and 1 to 3 nitrogen atom(s), for example, oxazolyl, isoxazolyl, oxadiazolyl (e.g.; 1,2,4-oxadiazolyl, 1,3,4-oxadiazolyl, 1,2,5-oxadiazolyl, etc.), etc.;
saturated 3 to 8-membered (more preferably 5 or 6-membered) heteromonocyclic group containing 1 or 2 oxygen atom(s) and 1 to 3 nitrogen atom(s), for example, morpholinyl, sydnonyl, morpholino, etc.;
unsaturated condensed heterocyclic group containing 1 or 2 oxygen atom(s) and 1 to 3 nitrogen atom(s), for example, benzoxazolyl, benzoxadiazolyl, etc.;
unsaturated 3 to 8-membered (more preferably 5 or 6-membered) heteromonocyclic group containing 1 or 2 sulfur atom(s) and 1 to 3 nitrogen atom(s), for example, thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.), dihydrothiazinyl, etc.;
saturated 3 to 8-membered (more preferably 5 or 6-membered) heteromonocyclic group containing 1 or 2 sulfur atom(s) and 1 to 3 nitrogen atom(s), for example thiazolidinyl, thiomorpholinyl, thiomorpholino, etc.;
unsaturated 3 to 8-membered (more preferably 5 or 6-membered) heteromonocyclic group containing 1 or 2 sulfur atom(s), for example, thienyl, dihydrodithiinyl, dihydrodithionyl, etc.;
unsaturated condensed heterocyclic group containing 1 or 2 sulfur atom(s) and 1 to 3 nitrogen atom(s), for example, benzothiazolyl, benzothiadiazolyl, imidazothiadiazolyl, etc.;
unsaturated 3 to 8-membered (more preferably 5 or 6-membered) heteromonocyclic group containing an oxygen atom, for example, furyl etc.;
saturated 3 to 8-membered (more preferably 5 or 6-membered) heteromonocyclic group containing 1 or 2 oxygen atom(s), for example, tetrahydrofuran, tetrahydropyran, dioxacyclopentane, dioxacyclohexane, etc.;
unsaturated 3 to 8-membered (more preferably 5 or 6-membered) heteromonocyclic group containing an oxygen atom and 1 or 2 sulfur atom(s), for example, dihydrooxathiinyl, etc.;
unsaturated condensed heterocyclic group containing 1 or 2 sulfur atom(s), for example benzothienyl, benzodithiinyl, etc.;
unsaturated condensed heterocyclic group containing an oxygen atom and 1 or 2 sulfur atom(s), for example, benzoxathiinyl, etc.;
saturated condensed heterocyclic group containing 1 to 3 nitrogen atom(s), for example, hexahydropyrrolopyrazinyl, etc.; and the like,
and this “heterocyclic group” may have one or more suitable substituent (s) selected from the group consisting of lower alkyl, oxo, cyclo(lower)alkyl, hydroxy(lower)alkyl, carboxy(lower)alkanoyl which may have amino and heterocycliccarbonyl.

Suitable example of “cyclo(lower)alkyl” may include cyclopropyl, cyclobutyl, cyclopentyl, cyclohexyl, and the like, and this “cyclo(lower)alkyl” may have one or more lower alkyl.

Suitable example of “cyclo(lower)alkyloxy” may include cyclopropyloxy, cyclobutyloxy, cyclopentyloxy, cyclohexyloxy, and the like.

Suitable example of “cyclo(higher)alkyl” may include cycloheptyl, cyclooctyl, cyclo-3,5-dimethyloctyl, cyclo-3,7-dimethyloctyl, cyclononyl, cyclodecyl, and the like.

Suitable example of “acyl group” may include aliphatic acyl, aromatic acyl, arylaliphatic acyl and heterocyclic-aliphatic acyl derived from carboxylic acid, carbonic acid, carbamic acid, sulfonic acid, and the like.

Suitable example of said “acyl group” may be illustrated as follows.
carboxy; carbamoyl; mono or di (lower)alkylcarbamoyl (e.g., methylcarbamoyl, dimethylcarbamoyl, ethylcarbamoyl, diethylcarbamoyl, etc.)

Aliphatic acyl such as lower or higher alkanoyl (e.g., formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, etc.);
lower or higher alkoxycarbonyl (e.g., methoxycarbonyl, ethoxycarbonyl, t-butoxycarbonyl, t-pentyloxycarbonyl, heptyloxycarbonyl, etc.); lower alkenyloxycarbonyl (e.g., vinyloxycarbonyl, propenyloxycarbonyl, allyloxycarbonyl, butenyloxycarbonyl, butedienyloxycarbonyl, pentenyloxycarbonyl, hexenyloxycarbonyl, etc.);
lower or higher alkylsulfonyl (e.g., methylsulfonyl, ethylsulfonyl, etc.);
lower or higher alkoxysulfonyl (e.g., methoxysulfonyl, ethoxysulfonyl, etc.); or the like;

Aromatic acyl such as
aroyl (e.g., benzoyl, toluoyl, naphthoyl, etc.);
ar(lower)alkanoyl [e.g., phenyl(C₁-C₆)alkanoyl (e.g., phenylacetyl, phenylpropanoyl, phenylbutanoyl, phenylisobutanoyl, phenylpentanoyl, phenylhexanoyl, etc.), naphthyl(C₁-C₆)alkanoyl (e.g., naphthylacetyl, naphthylpropanoyl, naphthylbutanoyl, etc.), etc.];
ar(lower)alkenoyl [e.g., phenyl(C₃-C₆)alkenoyl (e.g., phenylpropenoyl, phenylbutenoyl, phenylmethacryloyl, phenylpentanoyl, phenylhexenoyl, etc.), naphthyl(C₃-C₆)alkenoyl (e.g., naphthylpropenoyl, naphthylbutenoyl, etc.), etc.];
ar(lower)alkoxycarbonyl [e.g., phenyl(C₁-C₆)alkoxycarbonyl (e.g., benzyloxycarbonyl, etc.), fluorenyl(C₁-C₆)alkoxycarbonyl (e.g., fluorenylmethyloxycarbonyl, etc.), etc.];
aryloxycarbonyl (e.g., phenoxycarbonyl, naphthyloxycarbonyl, etc.);
aryloxy(lower)alkanoyl (e.g., phenoxyacetyl, phenoxypropionyl, etc.);
arylcarbamoyl (e.g., phenylcarbamoyl, etc.);
arylthiocarbamoyl (e.g., phenylthiocarbamoyl, etc.);
arylglyoxyloyl (e.g., phenylglyoxyloyl, naphthylglyoxyloyl, etc.);
arylsulfonyl which may have 1 to 4 lower alkyl (e.g., phenylsulfonyl, p-tolylsulfonyl, etc.);
aroyl (e.g., benzoyl) substituted with one or more suitable substituent(s); or the like;

Heterocyclic acyl such as
heterocycliccarbonyl;
heterocyclic(lower)alkanoyl (e.g., heterocyclicacetyl, heterocyclicpropanoyl, heterocyclicbutanoyl, heterocyclicpentanoyl, heterocyclichexanoyl, etc.);
heterocyclic(lower)alkenoyl (e.g., heterocyclicpropenoyl, heterocyclicbutenoyl, heterocyclicpentenoyl, heterocyclichexenoyl, etc.);
heterocyclicglyoxyloyl; or the like;
   in which suitable “heterocyclic” moiety in the terms “heterocycliccarbonyl”, “heterocyclic(lower)alkanoyl”, “heterocyclic(lower)alkenoyl” and “heterocyclicglyoxyloyl” can be referred to aforementioned “heterocyclic” moiety.

Suitable example of “acyl group” of R¹ can be referred to aforementioned “acyl group”, in which the preferred one may be lower alkoxycarbonyl and benzoyl substituted with one or more suitable substituent(s), and more preferred one may be
1) benzoyl substituted with heterocyclic group substituted with phenyl substituted with optionally substituted heterocyclic group,
2) benzoyl substituted with heterocyclic group substituted with heterocyclic group substituted with optionally substituted heterocyclic group,
3) benzoyl substituted with phenyl substituted with optionally substituted heterocyclic group,
4) benzoyl substituted with phenyl substituted with phenyl substituted with optionally substituted heterocyclic group,
   And the still more preferred one may be
1) benzoyl substituted with thiadiazolyl substituted with phenyl substituted with piperazinyl substituted with cyclo(lower)alkyl,
2) benzoyl substituted with thiadiazolyl substituted with phenyl substituted with piperazinyl substituted with cyclo(higher)alkyl,
3) benzoyl substituted with thiadiazolyl substituted with phenyl substituted with piperazinyl substituted with cyclo(lower)alkyl substituted with lower alkylidene,
4) benzoyl substituted with thiadiazolyl substituted with phenyl substituted with piperazinyl substituted with cyclo(lower)alkyl(lower)alkyl,
5) benzoyl substituted with thiadiazolyl substituted with phenyl substituted with piperazinyl substituted with lower alkoxy(higher)alkyl,
6) benzoyl substituted with thiadiazolyl substituted with phenyl substituted with piperidyl substituted with cyclo(lower)alkyl(lower)alkoxy,
7) benzoyl substituted with phenyl substituted with piperazinyl substituted with cyclo(lower)alkyl substituted with lower alkyl,
8) benzoyl substituted with thiadiazolyl substituted with phenyl substituted with hexahydropyrrolopyrazinyl,
9) benzoyl substituted with phenyl substituted with phenyl substituted with piperazinyl substituted with cyclo(lower)alkyl,
10) benzoyl substituted with thiadiazolyl substituted with phenyl substituted with piperazinyl substituted with cyclo(lower)alkyl substituted with lower alkyl,
11) benzoyl substituted with thiadiazolyl substituted with pyridyl substituted with piperazinyl substituted with cyclo(lower)alkyl substituted with lower alkyl,
12) benzoyl substituted with thiadiazolyl substituted with phenyl substituted with piperidyl substituted with lower alkoxy and cyclo(lower)alkyl,
   And the most preferred one may be
benzoyl substituted with thiadiazolyl substituted with phenyl substituted with piperazinyl substituted with cyclo(lower)alkyl.

Suitable example of “lower alkyl” in the term of “lower alkyl substituted with one or more hydroxy” can be referred to aforementioned “lower alkyl”, in which the preferred one may be methyl, ethyl, propyl, isopropyl, butyl, pentyl and hexyl.

Suitable example of “lower alkyl substituted with one or more hydroxy” may be dihydroxypropyl, dihydroxyisopropyl, trihydroxybutyl, tetrahydroxypentyl, pentahydroxyhexyl and diacetyloxyisopropyl.

Suitable example of “amino protective group” may be included in aforementioned “acyl group”, a conventional protective group such as ar(lower)alkoxycarbonyl and lower alkoxycarbonyl, in which the preferred one may be phenyl(C₁-C₄)alkoxycarbonyl and fluorenyl(C₁-C₄)alkoxycarbonyl and (C₁-C₄)alkoxycarbonyl, and the most preferred one may be benzyloxycarbonyl, fluorenylmethoxycarbonyl and tert-butoxycarbonyl.

Suitable example of “acyl” moiety of “acyloxy” can be referred to aforementioned “acyl group”, in which the preferred one may be lower alkenyloxycarbonyl, and the most preferred one may be allyloxycarbonyl.

Suitable example of “acyloxy” may be lower alkenyloxycarbonyloxy, and the more preferred one may be allyloxycarbonyloxy.

Particularly, the preferred examples of the lipopeptide compound (I) of the present invention may be
R¹ is benzoyl substituted with a suitable substituent,
R² is hydrogen,
R³ is hydrogen or lower alkyl substituted with two hydroxy,
R⁴ is hydrogen,
R⁵ is methoxy, and
R⁶ is hydroxy,
   And, the more preferred one may be
R¹ is
   1) benzoyl substituted with heterocyclic group substituted with phenyl substituted with optionally substituted heterocyclic group,
   2) benzoyl substituted with heterocyclic group substituted with heterocyclic group substituted with optionally substituted heterocyclic group,
   3) benzoyl substituted with phenyl substituted with optionally substituted heterocyclic group,
   4) benzoyl substituted with phenyl substituted with phenyl substituted with optionally substituted heterocyclic group,
R² is hydrogen,
R³ is hydrogen or lower alkyl substituted with two hydroxy,
R⁴ is hydrogen,
R⁵ is methoxy, and
R⁶ is hydroxy,
   And, the still more preferred one may be
R¹ is
   1) benzoyl substituted with thiadiazolyl substituted with phenyl substituted with piperazinyl substituted with cyclo(lower)alkyl,
   2) benzoyl substituted with thiadiazolyl substituted with phenyl substituted with piperazinyl substituted with cyclo(higher)alkyl,
   3) benzoyl substituted with thiadiazolyl substituted with phenyl substituted with piperazinyl substituted with cyclo(lower)alkyl substituted with lower alkylidene,
   4) benzoyl substituted with thiadiazolyl substituted with phenyl substituted with piperazinyl substituted with cyclo(lower)alkyl(lower)alkyl,
   5) benzoyl substituted with thiadiazolyl substituted with phenyl substituted with piperazinyl substituted with lower alkoxy(higher)alkyl,
   6) benzoyl substituted with thiadiazolyl substituted with phenyl substituted with piperidyl substituted with cyclo(lower)alkyl(lower)alkoxy,
   7) benzoyl substituted with phenyl substituted with piperazinyl substituted with cyclo(lower)alkyl substituted with lower alkyl,
   8) benzoyl substituted with thiadiazolyl substituted with phenyl substituted with hexahydropyrrolopyrazinyl,
   9) benzoyl substituted with phenyl substituted with phenyl substituted with piperazinyl substituted with cyclo(lower)alkyl,
   10) benzoyl substituted with thiadiazolyl substituted with phenyl substituted with piperazinyl substituted with cyclo(lower)alkyl substituted with lower alkyl,
   11) benzoyl substituted with thiadiazolyl substituted with pyridyl substituted with piperazinyl substituted with cyclo(lower)alkyl substituted with lower alkyl,
   12) benzoyl substituted with thiadiazolyl substituted with phenyl substituted with piperidyl substituted with lower alkoxy and cyclo (lower) alkyl,
R² is hydrogen,
R³ is hydrogen or lower alkyl substituted with two hydroxy,
R⁴ is hydrogen,
R⁵ is methoxy, and
R⁶ is hydroxy,
   And, the most preferred one may be
R¹ is benzoyl substituted with thiadiazolyl substituted with phenyl substituted with piperazinyl substituted with cyclo(lower)alkyl,
R² is hydrogen,
R³ is hydrogen or lower alkyl substituted with two hydroxy,
R⁴ is hydrogen,
R⁵ is methoxy, and
R⁶ is hydroxy.

The processes for preparing the lipopeptide compound (I) of the present invention are explained in detail in the following.

Process 1

The object compound (Ia) or a salt thereof can be prepared by reducing a compound (II) or a salt thereof, which is prepared by Preparations mentioned later or its similar method. Suitable salts of the compounds (Ia) and (II) may be the same as those exemplified for the compound (I).

The reaction can be carried out in a conventional manner namely, chemical reduction or catalytic reduction.

Suitable reducing agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, hydride transfer reagent such as aluminum hydride compound (e.g. lithium aluminum hydride, lithium hydridotri-t-butoxyaluminate, etc.), borohydride compound (e.g. sodium borohydride, sodium cyanoborohydride, etc.) or the like etc.].

Suitable catalysts to be used in catalytic reduction are conventional ones such as platinum catalyst [e.g. platinum plate, spongy platinum, platinum black, colloidal platinum, platinum oxide, platinum wire, etc.], palladium catalyst [e.g., spongy palladium, palladium black, palladium oxide, palladium on carbon, colloidal palladium, palladium on barium sulfate, palladium on barium carbonate, etc.], nickel catalyst [e.g. reduced nickel, nickel oxide, Raney nickel, etc.], cobalt catalyst [e.g. reduced cobalt, Raney cobalt, etc.], iron catalyst [e.g. reduced iron, Raney iron, etc.], copper catalyst [e.g. reduced copper, Raney copper, Ullman copper, etc.] or the like.

The reaction of this process is usually carried out in a solvent such as water, alcohol [e.g. methanol, ethanol, propanol, etc.], acetic acid, diethyl ether, dioxane, tetrahydrofuran, methylene chloride, etc. or a mixture thereof.

The reaction is preferably carried out under somewhat milder conditions such as under cooling to warming. It is included within the scope of the present invention that “hydroxy” in R⁴ may be reduced to “hydrogen” during the reaction.

Process 2

The object compound (Ib) or a salt thereof can be prepared by reacting the compound (Ia) or its reactive derivative at the amino group or a salt thereof with the compound (III) of the formula:
Rₐ³═O (III)
or its reactive derivative, or a salt thereof.

The reaction can be carried out by forming Schiff's base type imino between the compound (Ia) or its reactive derivative at the amino group or a salt thereof and the compound (III), and then reduction.

Suitable reducing agents to be used in reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, hydride transfer reagent such as aluminum hydride compound (e.g. lithium aluminum hydride, lithium hydridotri-t-butoxyaluminate, etc.), borohydride compound (e.g. sodium borohydride, sodium cyanoborohydride, etc.) or the like etc.].

The reaction is usually carried out in a conventional solvent such as water, acetone, dioxane, acetonitrile, chloroform, methylene chloride, ethylene chloride, tetrahydrofuran, ethyl acetate, N,N-dimethylformamide, pyridine or any other organic solvent which do not adversely affect the reaction, or the mixture thereof.

The reaction temperature is not critical, and the reaction is usually carried out under cooling to heating.

Process 3

The object compound (Id) or a salt thereof can be prepared by subjecting a compound (Ic) or a salt thereof to elimination reaction of the amino protective group.

This reaction is carried out in accordance with a conventional method such as hydrolysis, reduction or the like.

The hydrolysis is preferably carried out in the presence of a base or an acid including Lewis acid. Suitable base may include an inorganic base and an organic base such as an alkali metal [e.g. sodium, potassium, etc.], an alkaline earth metal [e.g. magnesium, calcium, etc.], the hydroxide or carbonate or bicarbonate thereof, trialkylamine [e.g. trimethylamine, triethylamine, etc.], picoline, 1,5-diazabicyclo[4.3.0]non-5-ene, 1,4-diazabicyclo[2.2.2]octane, 1,8-diazabicyclo[5.4.0]undec-7-ene, or the like.

Suitable acid may include an organic acid [e.g. formic acid, acetic acid, propionic acid, trichloroacetic acid, trifluoroacetic acid, etc.] and an inorganic acid [e.g. hydrochloric acid, hydrobromic acid, sulfuric acid, hydrogen chloride, hydrogen bromide, etc.]. The elimination using Lewis acid such as trihaloacetic acid [e.g. trichloroacetic acid, trifluoroacetic acid, etc.] or the like is preferably carried out in the presence of cation trapping agents [e.g. anisole, phenol, etc.].

The reaction is usually carried out in a solvent such as water, an alcohol [e.g. methanol, ethanol, etc.], methylene chloride, tetrahydrofuran, a mixture thereof or any other solvent which does not adversely influence the reaction. A liquid base or acid can be also used as the solvent. The reaction temperature is not critical and the reaction is usually carried out under cooling to warming.

The reduction method applicable for the elimination reaction may include chemical reduction and catalytic reduction.

Suitable reducing agents to be used in chemical reduction are a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium, sulfate, palladium on barium carbonate, etc.], nickel catalysts [e.g. reduced nickel, nickel oxide, Raney nickel, etc.], cobalt catalysts [e.g. reduced cobalt, Raney cobalt, etc], iron catalysts [e.g. reduced iron, Raney iron, etc], copper catalysts [e.g. reduced copper, Raney copper, Ullman copper, etc.] and the like.

The reduction is usually carried out in a conventional solvent which does not adversely influence the reaction such as water, methanol, ethanol, propanol, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to warming.

Process 4

The object compound (If) or a salt thereof can be prepared by reacting the compound (Ie) or its reactive derivative at the amino group or a salt thereof with the compound (IV) of the formula:
Rₐ¹—OH (IV)
(wherein Rₐ¹ is acyl group)
   or its reactive derivative or a salt thereof.

Suitable reactive derivative of the compound (IV) may include an acid halide, an acid anhydride, an activated amide, an activated ester, and the like. Suitable examples of the reactive derivatives may be an acid chloride; an acid azide; a mixed acid anhydride with an acid such as substituted phosphoric acid [e.g., dialkylphosphoric acid, phenylphosphoric acid, diphenylphosphoric acid, dibenzylphosphoric acid, halogenated phosphoric acid, etc.], dialkylphosphorous acid, sulfurous acid, thiosulfuric acid, sulfuric acid, sulfonic acid [e.g., methanesulfonic acid, etc.], aliphatic carboxylic acid [e.g., acetic acid, propionic acid, butyric acid, isobutyric acid, pivaric acid, pentanoic acid, isopentanoic acid, 2-ethylbutyric acid trichloroacetic acid, etc.]; or aromatic carboxylic acid [e.g., benzoic acid, etc.]; a symmetrical acid, anhydride; an activated amide with imidazole, 4-substituted imidazole, dimethylpyrazole, triazole, tetrazole or 1-hydroxy-1H-benzotriazole; or an activated ester [e.g., cyanomethyl ester, methoxymethyl ester, vinyl ester, propargyl ester, p-nitrophenyl ester, 2,4-dinitrophenyl ester, trichlorophenyl ester, pentachloropentyl ester, mesylphenyl ester, phenylazophenyl ester, phenyl thioester, p-nitrophenyl thioester, p-cresyl thioester, carboxymethyl thioester, pyranyl ester, pyridyl ester, piperidyl ester, 8-quinolyl thioester, etc.], or an ester with a N-hydroxy compound [e.g. N,N-dimethylhydroxylamine, 1-hydroxy-2-(1H)-pyridone, N-hydroxysuccinimide, N-hydroxyphthalimide, 1-hydroxy-1H-benzotriazole, etc.], and the like. These reactive derivatives can optionally be selected from them according to the kind of the compound (IV) to be used.

Suitable salts of the compound (IV) and its reactive derivative can be referred to the ones as exemplified for the lipopeptide compound (I).

The reaction is usually carried out in a conventional solvent such as water, alcohol [e.g., methanol, ethanol, etc.], acetone, dioxane, acetonitrile, chloroform, methylene chloride, ethylene chloride, tetrahydrofuran, ethyl acetate, N,N-dimethylformamide, pyridine or any other organic solvent which does not adversely influence the reaction. These conventional solvent may also be used in a mixture with water.

In this reaction, when the compound (IV) is used in a free acid form or its salt form, the reaction is preferably carried out in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl)carbodiimide; N,N′-diethylcarbodiimide; N,N′-diisopropylcarbodiimide; N-ethyl-N′-(3-dimethylaminopropyl)carbodiimide; N-ethyl-N′-(3-dimethylaminopropyl)carbodiimide hydrochloride; N,N-carbonylbis-(2-methylimidazole); pentamethyleneketene-N-cyclohexylimine; diphenylketene-N-cyclohexylimine, ethoxyacetylene; 1-alkoxy-2-chloroethylene; trialkyl phosphite; ethyl polyphosphate; isopropyl polyphosphate; phosphorus oxychloride (phosphoryl chloride); phosphorus trichloride; thionyl chloride; oxalyl chloride; lower alkyl haloformate [e.g., ethyl chloroformate, isopropyl chloroformate, etc.]; triphenylphosphine; 2-ethyl-7-hydroxybenzisoxazolium salt; 2-ethyl-5-(m-sulfophenyl)isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, trichloromethyl chloroformate, phosphorous oxychloride, methanesulfonyl chloride, etc.; or the like.

The reaction may also be carried out in the presence of an inorganic or organic base such as an alkali metal carbonate, alkali metal bicarbonate, tri(lower)alkylamine (e.g., triethylamine, diisopropylethylamine, etc.), pyridine, di(lower)alkylaminopyridine (e.g., 4-dimethylaminopyridine, etc.), N-(lower)alkylmorpholine, N,N-di(lower)alkylbenzylamine, or the like.

The reaction temperature is not critical, and the reaction is usually carried out under cooling to warming.

The compounds obtained by the above Processes 1 to 4 can be isolated and purified by a conventional method such as pulverization, recrystallization, column-chromatography, high-performance liquid chromatography (HPLC), reprecipitation, desalting resin column chromatography, or the like.

The compounds obtained by the above Processes 1 to 4 may be obtained as its solvate (e.g., hydrate, ethanolate, etc.), and its solvate (e.g., hydrate, ethanolate, etc.) is included within the scope of the present invention.

It is to be noted that each of the lipopeptide compound (I) may include one or more stereoisomer such as optical isomer(s) and geometrical isomer(s) due to asymmetric carbon atom(s) and double bond(s) and all such isomers and the mixture thereof are included within the scope of the present invention.

The lipopeptide compound (I) or a salt thereof may include solvated compound [e.g., hydrate, ethanolate, etc.].

The lipopeptide compound (I) or a salt thereof may include both its crystal form and non-crystal form.

It should be understood that the lipopeptide compound (I) of the present invention may include the prodrug form.

The patent applications and publications cited herein are incorporated by reference.

In order to show the usefulness of the lipopeptide compound (I) of the present invention, the biological data of the representative compound is explained in the following.

### Biological Property of the Lipopeptide Compound (I) of the Present Invention

Test (Antimicrobial Activity):

In vitro antimicrobial activity of the object compound of Examples 1, 7, 12 and 19 disclosed later was determined by MICₛ in mouse serum as described below.

Test Method:

The MIC₅ in mouse serum were determined by the microdilution method using ICR mouse serum buffered with 20 mM HEPES buffer (pH 7.3) as a test medium. Inoculum suspension of 10⁶ cells/ml were prepared by a hemocytometric procedure and diluted to obtain an inoculum size of approximately 1.0×10³ cells/ml. Microplates were incubated at 37° C. for 0.24 hours in 5% CO₂. The MICₛ was defined as the lowest concentrations at which no visible growth was observed.

Test Result:

MIC (μG/ML)

| | Test organism |
|---|---|
| Test compound | Candida albicans FP-633 |
| The object compound of | <0.2 |
| Example 1 | |
| The object compound of | <0.2 |
| Example 7 | |
| The object compound of | <0.2 |
| Example 12 | |
| The object compound of | <0.2 |
| Example 19 | |

From the test result, it is realized that the lipopeptide compound (I) of the present invention has an antimicrobial activity (especially, antifungal activity).

In more details, the lipopeptide compound (I) of the present invention has an antifungal activity, particularly against the following fungi.
Acremonium;
Absidia (e.g., Absidia corymbifera, etc);
Aspergillus (e.g., Aspergillus clavatus, Aspergilius flavus, Aspergillus fumigatus, Aspergillus nidulans, Aspergillus niger, Aspergillus terreus, Aspergillus versicolor, etc); Blastomyces (e.g., Blastomyces dermatitidis, etc);
Candida (e.g., Candida albicans, Candida glabrata, Candida guilliermondii, Candida kefyr, Candida krusei, Candida parapsilosis, Candida stellatoidea, Candida tropicalis, candida utilis, etc.);
Cladosporium (e.g., Cladosporium trichloides, etc);
Coccidioides (e.g., Coccidioides immitis, etc);
Cryptococcus (e.g., Cryptococcus neoformans, etc);
Cunninghamella (e.g., Cunninghamella elegans, etc);
Dermatophyte;
Exophiala (e.g., Exophiala dermatitidis, Exophiala spinifera, etc);
Epidermophyton (e.g., Epidermophyton floccosum, etc);
Fonsecaea (e.g., Fonsecaea pedrosoi, etc);
Fusarium (e.g., Fusarium solani, etc);
Geotrichum (e.g., Geotrichum candiddum, etc);
Histoplasma (e.g., Histoplasma capsulatum var. capsulatum, etc)
Malassezia (e.g., Malassezia furfur, etc);
Microsporum (e.g., Microsporum canis, Microsporum gypseum, etc);
Mucor;
Paracoccidioides (e.g., Paracoccidioides brasiliensis, etc);
Penicillium (e.g., Penicillium marneffei, etc);
Phialophora;
Pneumocystis (e.g., Pneumocystis carinii, etc);
Pseudallescheria (e.g., Pseudallescheria boydii, etc);
Rhizopus (e.g., Rhizopus microsporus var. rhizopodiformis, Rhizopus oryzae, etc);
Saccharomyces (e.g., Saccharomyces cerevisiae, etc);
Scopulariopsis;
Sporothrix (e.g., Sporothrix schenckii, etc);
Trichophyton (e.g., Trichophyton mentagrophytes, Trichophyton rubrum, etc);
Trichosporon (e.g., Trichosporon asahii, Trichosporon cutaneum, etc).

The above fungi are well-known to cause various infection diseases in skin, eye, hair, nail, oral mucosa, gastrointestinal tract, bronchus, lung, endocardium, brain, meninges, urinary organ, vaginal protion, oral cavity, ophthalmus, systemic, kidney, bronchus, heart, external auditory canal, bone, nasal cavity, paranasal cavity, spleen, liver, hypodermal tissue, lymph doct, gastrointestine, articulation, muscle, tendon, interstitial plasma cell in lung, blood, and so on.

Therefore, the lipopeptide compound (I) of the present invention are useful for preventing and treating various infectious diseases, such as dermatophytosis (e.g., trichophytosis, etc), pityriasis versicolor, candidiasis, cryptococcosis, geotrichosis, trichosporosis, aspergillosis, penicilliosis, fusariosis, zygomycosis, sporotrichosis, chromomycosis, coccidioidomycosis, histoplasmosis, blastomycosis, paracoccidioidomycosis, pseudallescheriosis, mycetoma, mycotic keratitis, otomycosis, pneumocystosis, fungemia, and so on.

The combination use of azoles such as fluconazole, voriconazole, itraconazole, ketoconazole, miconazole, ravuconazole and posaconazole; polyenes such as amphotericin B, nystatin, liposamal and lipid forms thereof such as Abelcet, AmBisome, and Amphocil; purine or pyrimidine nucleotide inhibitors such as flucytosine; or polyxins such as nikkomycines, in particular nikkomycine Z or nikkomycine X; other chitin inhibitors; elongation factor inhibitors such as sordarin and analogs thereof; mannan inhibitors such as predamycin, bactericidal/permeability-inducing (BPI) protein products such as XMP.97 or XMP.127; or complex carbohydrate antifungal agents such as CAN-296; with the lipopeptide compound (I) or salt thereof is effective against above diseases.

The combination use of immunosuppressant such as tacrolimus, or G-CSF (Granulocyte-colony stimulating factor) with the lipopeptide compound (I) or a salt thereof is effective against above infectious diseases.

The pharmaceutical composition of the present invention can be used in the form of a pharmaceutical preparation, for example, in solid, semisolid or liquid form, which contains the lipopeptide compound (I) or a pharmaceutically acceptable salt thereof, as an active ingredient in admixture with an organic or inorganic carrier or excipient which is suitable for rectal; pulmonary (nasal or buccal inhalation); ocular; external (topical); oral administration; parenteral (including subcutaneous, intravenous and intramuscular) administrations; insufflation (including aerosols from metered dose inhalator); nebulizer; or dry powder inhalator.

The active ingredient may be compounded, for example, with the usual non-toxic, pharmaceutically acceptable carriers in a solid form such as granules, tablets, dragees, pellets, troches, capsules, or suppositories; creams; ointments; aerosols; powders for insufflation; in a liquid form such as solutions, emulsions, or suspensions for injection; ingestion; eye drops; and any other form suitable for use. And, if necessary, there may be included in the above preparation auxiliary substance such as stabilizing, thickening, wetting, emulsifying and coloring agents; perfumes or buffer; or any other commonly may be used as additives.

The lipopeptide compound (I) or a pharmaceutically acceptable salt thereof is/are included in the pharmaceutical composition in an amount sufficient to produce the desired antimicrobial effect upon the process or condition of diseases.

For applying the composition to humans, it is preferable to apply it by intravenous, intramuscular, pulmonary, oral administration, eye drop administration or insufflation. While the dosage of therapeutically effective amount of the lipopeptide compound (I) varies from and also depends upon the age and condition of each individual patient to be treated, in the case of intravenous administration, a daily dose of 0.01-400 mg of the lipopeptide compound (I) per kg weight of human being in the case of intramuscular administration, a daily dose of 0.1-20 mg of the lipopeptide compound (I) per kg weight of human being, in case of oral administration, a daily dose of 0.5-50 mg of the lipopeptide compound (I) per kg weight of human being is generally given for treating or preventing infectious diseases.

Especially in case of the treatment of prevention of Pneumocystis carinii infection, the followings are to be noted.

For administration by inhalation, the compounds of the present invention are conveniently delivered in the form of an aerosol spray presentation form pressurized as powders which may be formulated and the powder compositions may be inhaled with the aid of an insufflation powder inhaler device. The preferred delivery system for inhalation is a metered dose inhalation aerosol, which may be formulated as a suspension or solution of compound in suitable propellants such as fluorocarbons or hydrocarbons.

Because of desirability to directly treat lung and bronchi, aerosol administration is a preferred method of administration. Insufflation is also a desirable method, especially where infection may have spread to ears and other body cavities.

Alternatively, parenteral administration may be employed using drip intravenous administration.

For administration by intravenous administration, the preferred pharmaceutical composition is the lyophilized form containing the lipopeptide compound (I) or its pharmaceutically acceptable salt.

The amount of the lipopeptide compound (I) or its pharmaceutically acceptable salt contained in the composition for a single unit dosage of the present invention is 0.1 to 400 mg, more preferably 1 to 200 mg, still more preferably 5 to 100 mg, specifically 5, 10, 15, 20, 25, 30, 35, 40, 45, 50, 55, 60, 70, 75, 80, 85, 90, 95 and 100 mg.

The present invention further provides the following ones.

An article of manufacture, comprising packaging material and the compound (I) identified in the above contained within said packaging material, wherein said the compound (I) is therapeutically effective for preventing or treating infectious diseases caused by pathogenic microorganism, and wherein said packaging material comprises a label or a written material which indicates that said compound (I) can or should be used for preventing or treating infectious diseases caused by pathogenic microorganism.

A commercial package comprising the pharmaceutical composition containing the compound (I) identified in the above and a written matter associated therewith, wherein the written matter states that the compound (I) can or should be used for preventing or treating infectious diseases caused by pathogenic microorganism.

The following Preparations and Examples are given for the purpose of illustrating the present invention in more detail.

Preparation 1

A mixture of ethyl 4-fluorobenzoate (5 g), octahydropyrrolo[1,2-a]pyrazine (4.13 g) and potassium carbonate (4.93 g) in dimethylsulfoxide (25 ml) was stirred for 4 hours at 150° C. The reaction mixture was poured into water (250 ml). The resulting precipitate was collected by filtration, and dried under reduced pressure to give ethyl 4-[hexahydropyrrolo[1,2-a]pyrazin-2(1H)-yl]benzoate (5.97 g).

IR(KBr): 1695.1, 1602.6, 1517.7, 1288.2, 1239.8, 1187.9 cm−1

MS(ESI+) m/z: 275.4 (M+H)

NMR(CDCl₃, δ) 1.37(3H, t, J=7.1 Hz), 1.69-3.89(13H, m), 4.33(2H, q, J=7.1 Hz), 6.84-6.91(2H, m), 7.89-7.96(2H, m)

Preparation 2

A solution of ethyl 4-[hexahydropyrrolo[1,2-a]pyrazin-2(1H)-yl]benzoate (5.9 g) and hydrazine monohydrate (21 ml) in ethanol (35 ml) was stirred at reflux for 14 hours. The reaction mixture was poured into water (56 ml). The resulting precipitate was collected by filtration, and dried under reduced pressure to give 4-[hexahydropyrrolo[1,2-a]pyrazin-2(1H)-yl]benzohydrazide (4.71 g).

IR(KBr): 3326.6, 1660.4, 1612.2, 1602.6, 1510.0, 1332.6, 1245.8 cm−1

MS(ESI+) m/z: 261.2(M+H), 283.3(M+Na)

NMR(CDCl₃, δ): 1.45-3.90(13H, m), 4.06(2H, brs), 6.86-6.94(2H, m), 7.26(1H, brs), 7.62-7.69(2H, m)

Preparation 3

To a solution of 4-[hexahydropyrrolo[1,2-a]pyrazin-2(1H)-yl]benzohydrazide (4.06 g) and pyridine (3.8 ml) in tetrahydrofuran (70 ml) was added methyl 4-(chlorocarbonyl)benzoate (3.1 g) under ice-cooling, and the mixture was stirred at ambient temperature for 4 hours. The reaction mixture was poured into water (70 ml). The resulting precipitate was collected by filtration, and dried under reduced pressure to give methyl 4-[2-[4-[hexahydropyrrolo[1,2-a]pyrazin-2(1H)-yl]-benzoyl]hydrazinocarbonyl]benzoate (4.6 g).

IR(KBr): 3207.0, 1714.4, 1637.3, 1606.4, 1280.5 cm−1

MS(ESI+) m/z: 423.3(M+H)

NMR(DMSO-d₆, δ): 1.60-4.01(16H, m), 6.90-7.15(2H, m), 7.84-8.12(6H, m), 10.33(1H, s), 10.61(1H, s)

Preparation 4

A mixture of methyl 4-[2-[4-[hexahydropyrrolo[1,2-a]pyrazin-2(1H)-yl]benzoyl]-hydrazinocarbonyl]benzoate (4.3 g) and phosphorus pentasulfide (2.94 g) in pyridine (65 ml) was refluxed over night. The reaction mixture was poured into water (130 ml). The mixture was adjusted to pH 9 with 1N-sodiumhydroxide solution. The resulting precipitate was collected by filtration, and washed acetonitrile (100 ml). The precipitate was collected by filtration, and dried under reduced pressure to give methyl 4-[5-[4-[hexahydropyrrolo[1,2-a]pyrazin-2(1H)-yl]phenyl]-1,3, 4-thiadiazol-2-yl]benzoate (3.47 g).

IR(KBr): 1714.4, 1604.5, 1438.6, 1413.6, 1276.6 cm−1

MS(ESI+) m/z: 421.1 (M+H)

NMR(CDCl₃, δ): 1.60-4.04 (16H, m), 7.08-7.13 (2H, m) 7.83-7.88(2H, m), 8.13(4H, s)

Preparation 5

A mixture of methyl 4-[5-[4-[hexahydropyrrolo[1,2-a]pyrazin-2(1H)-yl]phenyl]-1,3, 4-thiadiazol-2-yl]benzoate (3.3 g) and sodiumhydroxide (0.628 g) in a mixture of water (12 ml) and tetrahydrofuran (50 ml) was refluxed for 5.5 hours. The reaction mixture was poured into water (50 ml). The mixture was adjusted to pH 1-2 with 1N-hydrochloric acid. The resulting precipitate was collected by filtration, and washed tetrahydrofuran, and dried under reduced pressure to give 4-[5-[4-[hexahydropyrrolo[1,2-a]pyrazin-2(1H)-yl]phenyl]-1,3, 4-thiadiazol-2-yl]benzoic acid (3.09 g).

IR(KBr): 3016.1, 1704.8, 1606.4, 1440.6, 1413.6 cm−1

MS(ESI−) m/z: 405.2(M−H)

Preparation 6

A mixture of 4-[5-[4-hexahydropyrrolo[1,2-a]pyrazin-2(1H)-yl]phenyl]-1,3,4-thiadiazol-2-yl]benzoic acid (3.0 g), O-(benzotriazol-1-yl)-N,N,N′,N′-tetramethyluronium hexafluorophosphate (3.36 g) and N,N-diisopropylethylamine (2.57 ml) in N-Methylpyrrolidone (30 ml) was stirred at ambient temperature over night. The reaction mixture was poured into water (240 ml). The resulting precipitate was collected by filtration, and washed tetrahydrofuran, and dried under reduced pressure to give 1-[4-[5-[4-[hexahydropyrrolo[1,2-a]pyrazin-2(1H)-yl]phenyl]-1,3,4-thiadiazol-2-yl]benzoyloxy]-1H-1,2,3-benzotriazole (3.33 g).

IR(KBr): 1778.0, 1602.6, 1440.6, 1413.6, 1234.2 cm−1

MS(ESI+) m/z: 524.2(M+H)

NMR(DMSO-d₆, δ): 1.40-4.40(13H, m), 7.11-7.15(2H, m), 7.37-8.49(10H, m)

Preparation 7

A mixture of 1-cyclohexylpiperazine (1.35 g), 4,4′-dibromo-1-1′-biphenyl (5.01 g), palladium(II)acetate (0.288 g), racemic-2,2′-bis(diphenylphosphino)-1,1′-binaphthyl (0.799 g) and cesium carbonate (7.31 g) in toluene (32 ml) was stirred at 100° C. for 2.5 hours. The reaction mixture was diluted with dichloromethane (150 ml). The suspension was filtered through celite, and the filtrate was evaporated under reduced pressure. The residue was purified by column chromatography over silica using dichloromethane/methanol (10:1) as the elution to give 1-(4′-bromo-1,1′-biphenyl-4-yl)-4-cyclohexylpiperazine (2.74 g).

IR(KBr): 1606.4, 1481.1, 1446.4, 1236.1 cm−1

MS(ESI+) m/z: 399.2(M+H)

NMR(CDCl₃, δ): 1.08-3.28(19H, m), 6.94-7.00(2H, m), 7.35-7.53(6H, m)

Preparation 8

A mixture of 4-(ethoxycarbonyl)phenylboronic acid (1.15 g), 1-(4′-bromo-1,1′-biphenyl-4-yl)-4-cyclohexylpiperazine (2.37 g), sodium carbonate (1.32 g) and tetrakis(triphenylphosphine)palladium(0) (0.343 g) in a mixture of 1,2-dimethoxyethane (46 ml) and water (7 ml) was stirred at 80° C. for 6.5 hours. The reaction mixture was poured into water (460 ml). The resulting precipitate was collected by filtration, and washed 2-propanol, and dried under reduced pressure to give ethyl 4″-(4-cyclohexyl-1-piperazinyl)-1,1′:4′,1″-terphenyl-4-carboxylate (1.88 g).

IR(KBr): 1714.4, 1602.6, 1486.8, 1448.3, 1274.7 cm−1

MS(ESI+) m/z: 469.3(M+H)

NMR(CDCl₃, δ): 1.10-3.30(22H, m), 4.41(2H, q, J=7.1 Hz), 6.99-7.03(2H, m), 7.44-7.75(8H, m), 8.10-8.16(2H, m)

Preparation 9

A mixture of ethyl 4″-(4-cyclohexyl-1-piperazinyl)-1,1′:4′,1″-terphenyl-4-carboxylate (1.87 g) and sodiumhydroxide (0.319 g) in a mixture of water (6.2 ml), ethanol (28 ml) and tetrahydrofuran (28 ml) was stirred at reflux for 4 hours. The reaction mixture was poured into water (120 ml). The mixture was adjusted to pH 1-2 with 1N-hydrochloric acid. The resulting precipitate was collected by filtration, and washed tetrahydrofuran, and dried under reduced pressure to give 4″-(4-cyclohexyl-1-piperazinyl)-1,1′:4′,1″-terphenyl-4-carboxylic acid (1.69 g).

IR(KBr): 3228.3, 1699.0, 1600.6, 1450.2, 1396.2 cm−1

MS(ESI−) m/z: 439.4(M−H)

Preparation 10

A mixture of 4″-(4-cyclohexyl-1-piperazinyl)-1,1′:4′,1″-terphenyl-4-carboxylic acid (1.58 g), O-(benzotriazol-1-yl)-N,N,N′,N′-tetramethyluronium hexafluorophosphate (1.63 g) and N,N-diisopropylethylamine (1.25 ml) in N-Methylpyrrolidone (16 ml) was stirred at ambient temperature over night. The reaction mixture was poured into water (130 ml). The resulting precipitate was collected by filtration, and washed diisopropylether, and dried under reduced pressure to give 1-[4″-(4-cyclohexyl-1-piperazinyl)-1,1′:4′,1″-terphenyl-4-yl]carbonyloxy-1H-1,2,3-benzotriazole (1.65 g).

IR(KBr): 1778.0, 1598.7, 1490.7, 1448.3, 1220.7 cm−1

MS(ESI−) m/z: 556.5(M−H)

Preparation 11

A solution of ethyl 4-(1-piperazinyl)benzoate (9.75 g), cyclopentanecarboxyaldehyde (4.9 g) and acetic acid (7.5 g) in methanol (50 ml) and methylene chloride (50 ml) was cooled with an ice bath and treated with sodium acetoxyborohydride (17.6 g) in portions over 30 minutes. After stirring for 1 hour at the same temperature and 15 hours at room temperature, the reaction mixture was diluted with ethyl acetate, washed with saturated sodium hydrogen carbonate solution, water, brine and the organic layer dried over magnesium sulfate, evaporated and the crude residue purified by silica-gel column chromatography, eluting with methylene chloride-methanol mixtures to afford ethyl 4-[4-(cyclopentylmethyl)-1-piperazinyl]benzoate (8.07 g) as a colorless solid.

MS(ESI+) m/z: 317.4(M+H)

NMR (CDCl₃, δ): 1.1-1.33(2H, m), 1.36(3H, t, J=7.1 Hz), 1.4-1.9(6H, m), 2.0-2.2(1H, m), 2.31(2H, d, J=7.3 Hz), 2.54-2.59(4H, m), 3.3-3.35(4H, m), 4.32(2H, q, J=7.1 Hz), 6.86(2H, d, J=9 Hz), 7.92(2H, d, J=9 Hz)

The following compound was obtained according to a similar manner to that of Preparation 2.

Preparation 12

4-[4-(cyclopentylmethyl)piperazinyl]benzohydrazide

MS(ESI+) m/z: 303.4(M+H)

NMR (DMSO-d₆, δ): 1.18-1.21(2H, m), 1.49-1.56(4H, m), 1.68-1.70(2H, m), 2.05-2.15(1H, m), 2.22(2H, d, J=7.5 Hz), 2.46-2.50(4H, m), 3.21-3.24(4H, m), 4.35(2H, s), 6.92(2H, d, J=8.9 Hz), 7.6.9(2H, d, J=8.9 Hz), 9.46(1H, s)

The following compound was obtained according to a similar manner to that of Preparation 3.

Preparation 13

Methyl 4-[2-[4-[4-(cyclopentylmethyl)piperazinyl]-benzoyl]hydrazinocarbonyl]benzoate

MS(ESI+) m/z: 463.6(M+H)

NMR (DMSO-d₆, δ): 1.15-1.24(2H, m), 1.4-1.8(6H, m) 2.03-2.17(1H, m), 2.24(2H, d, J=7.3 Hz), 2.48-2.52(4H, m), 3.2-3.35(4H, m), 3.90(3H, s), 6.99(2H, d, J=8.9 Hz), 7.81(2H, d, J=8.9 Hz), 8.02(2H, d, J=8.7 Hz), 8.09(2H, d, J=8.7 Hz), 10.25(1H, s), 10.57(1H, s)

The following compound was obtained according to a similar manner to that of Preparation 4.

Preparation 14

Methyl 4-[5-[4-[4-(cyclopentylmethyl)piperazinyl]-phenyl]-1,3,4-thiadiazol-2-yl]benzoate

MS(ESI+) m/z: 463.53(M+H)

NMR (CDCl₃, δ): 1.1-1.35(2H, m), 1.4-1.9(6H, m), 2.0-2.2(1H, m), 2.35(2H, d, J=7.2 Hz), 2.6-2.65(4H, m), 3.34-3.39(4H, m), 3.96(3H, s), 6.95(2H, d, J=9 Hz), 7.89(2H, d, J=9 Hz), 8.04-8.17(4H, m)

The following compound was obtained according to a similar manner to that of Preparation 5.

Preparation 15

4-[5-[4-[4-(cyclopentylmethyl)piperazinyl]phenyl]-1,3,4-thiadiazol-2-yl]benzoic acid

MS(ESI+) m/z: 449.2(M−Cl)

NMR (DMSO-d₆, δ) 1.2-2.5(10H, m), 2.5-4.0(10H, m), 7.16(2H, d, J=8.7 Hz), 7.92(2H, d, J=8.7 Hz), 8.12(4H, s)

The following compound was obtained according to a similar manner to that of Preparation 6.

Preparation 16

1-[4-[5-[4-[4-(cyclopentylmethyl)piperazinyl]phenyl]-1,3,4-thiadiazol-2-yl]benzoyloxy]-1H-1,2,3-benzotriazole

IR(KBr): 1776.1 cm−1

The Starting Compounds used and the Object Compounds obtained in the following Preparation 17 and 18 are given in the table as below, in which the formulas of the starting compounds are in the upper column, and the formulas of the object compounds are in the lower column, respectively.

| Preparation No. | Formula |
|---|---|
| 17 | 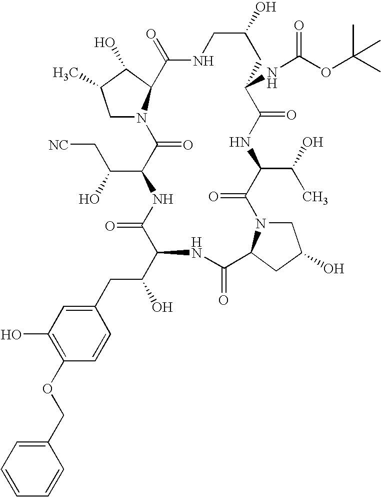 |
| | 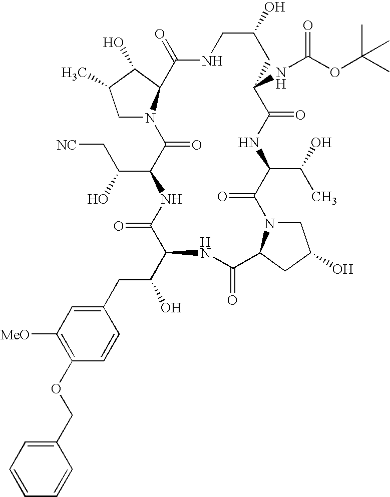 |
| 18 | 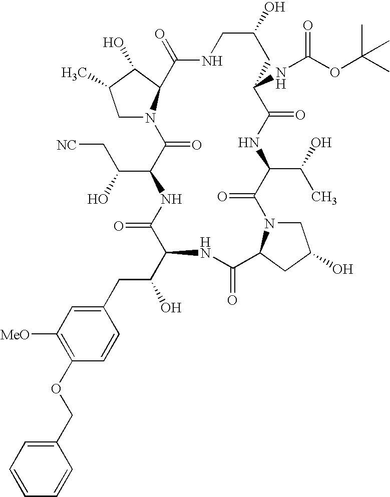 |
| | 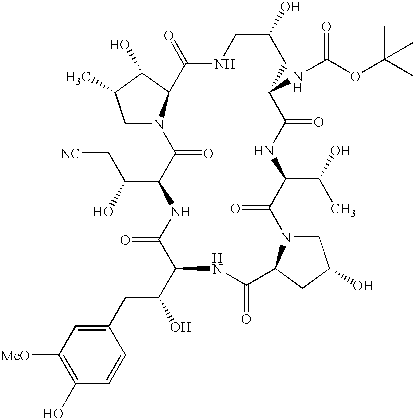 |

Preparation 17

A mixture of starting compound (17) (10 g) and lithium hydroxide monohydrate (0.44 g) in N,N-dimethylformamide (100 ml) was stirred for 2 hours at room temperature. To the reaction mixture methyl iodide (1.9 ml) was added. After stirring for 22 hours at room temperature, the reaction mixture was poured into a mixture of acetonitrile (0.8 L) and water (1.2 L) The solution was subjected to column chromatography on ODS (Daiso-gel, SP-120-40/60-ODS-B) (1 L) eluting with 40% acetonitrile in water. The eluted fractions containing the desired product were collected and evaporated in vacuo. The residue was lyophilized to give object compound (17) (7.65 g).

NMR (DMSO-d₆+D₂O, δ): 0.96 (3H, d, J=6.8 Hz), 1.08 (3H, d, J=5.6 Hz), 1.67 (9H, s), 1.2-2.9 (9H, m), 3.1-3.3 (1H, m), 3.5-4.5 (15H, m), 3.75 (3H, s), 4.8-4.9 (2H, m), 5.04 (2H, s), 6.6-7.0 (3H, m), 7.3-7.5 (5H, m)

MS (ESI+) m/z: 1033.3 (M+Na)

Preparation 18

A mixture of starting compound (18) (38.4 g) and 10% palladium on carbon (50% wet) (7.7 g) in methanol (400 ml) was stirred at room temperature under hydrogen atmosphere for 5 hours. After removal of insoluble solids, the filtrate was concentrated in vacuo to give object compound (18) (35.2 g).

NMR (DMSO-d₆+D₂O, δ): 0.95 (3H, d, J=6.8 Hz), 1.08 (3H, d, J=5.7 Hz), 1.36 (9H, s), 1.2-2.9 (9H, m), 3.1-3.3 (1H, m), 3.4-4.5 (15H, m), 3.74 (3H, s), 4.8-4.9 (2H, m), 6.5-6.7 (3H, m)

MS (ESI+) m/z: 943.3 (M+Na)

The Starting Compounds used and the Object Compounds obtained in the following Examples 1 to 25 are given in the table as below, in which the formulas of the starting compounds are in the upper column, and the formulas of the object compounds are in the lower column, respectively.

| Example | |
|---|---|
| No. | Formula |
| 1 | 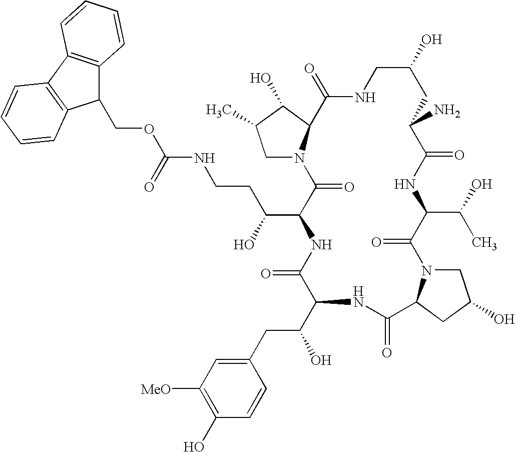 |
| | 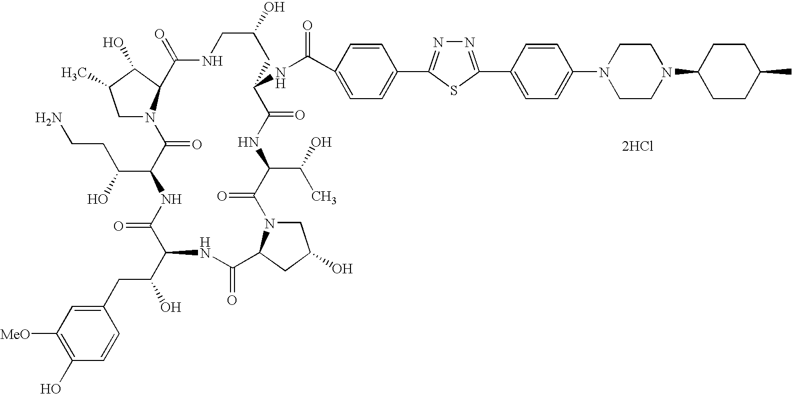 |
| 2 | 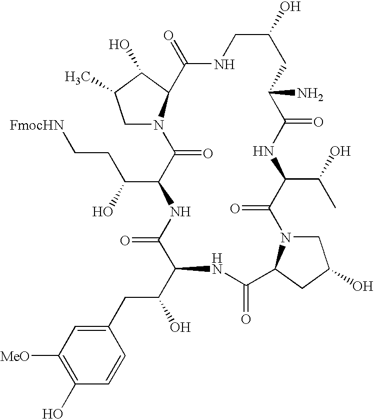 |
| | 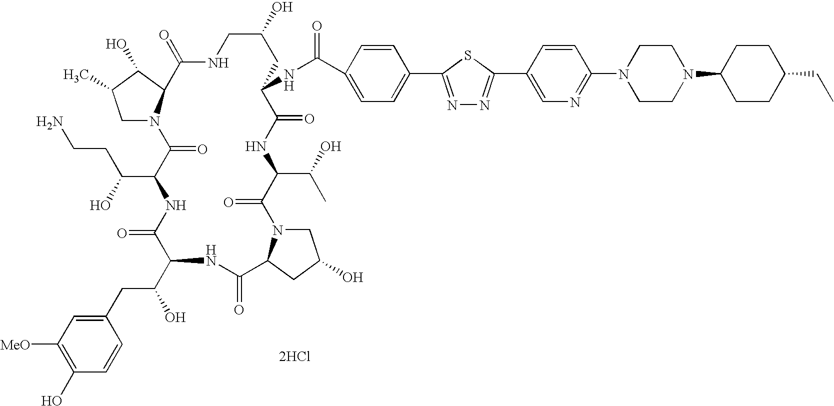 |
| 3 | 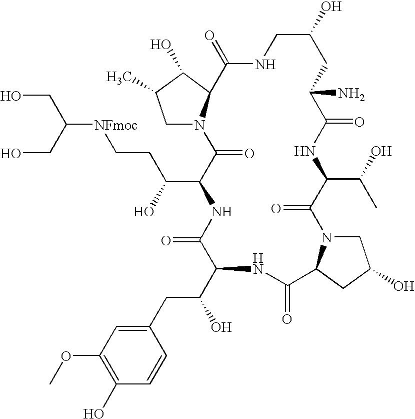 |
| | 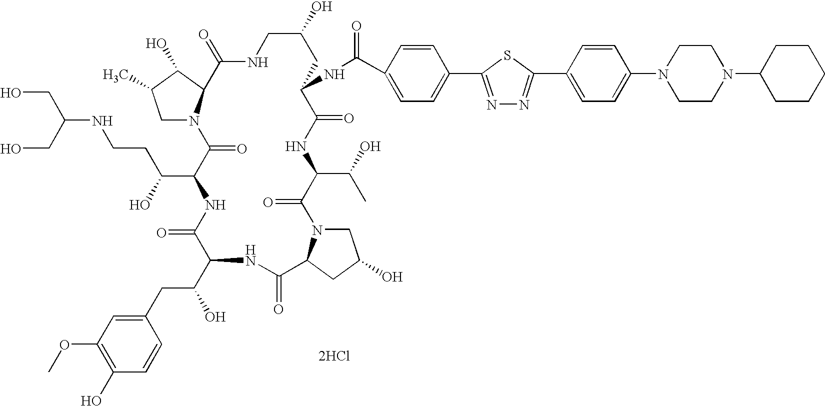 |
| 4 | 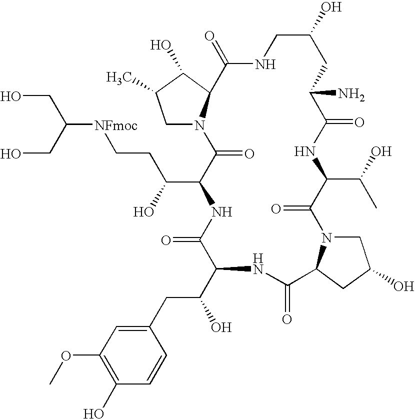 |
| | 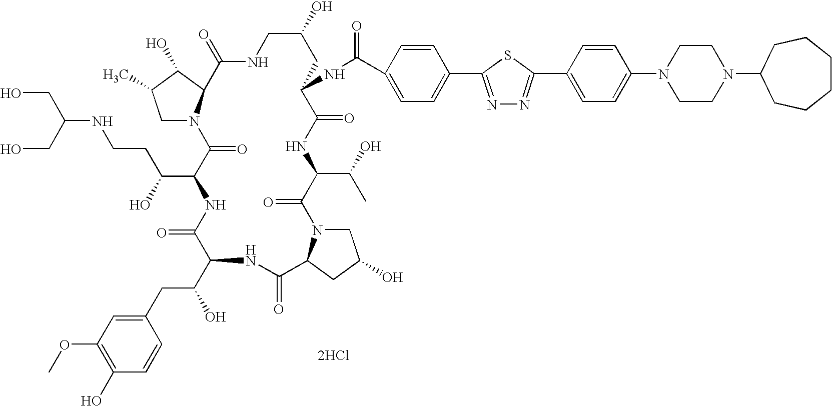 |
| 5 | 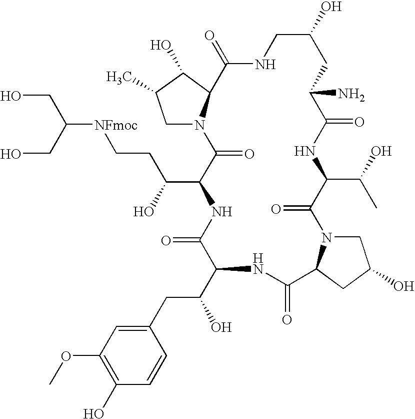 |
| | 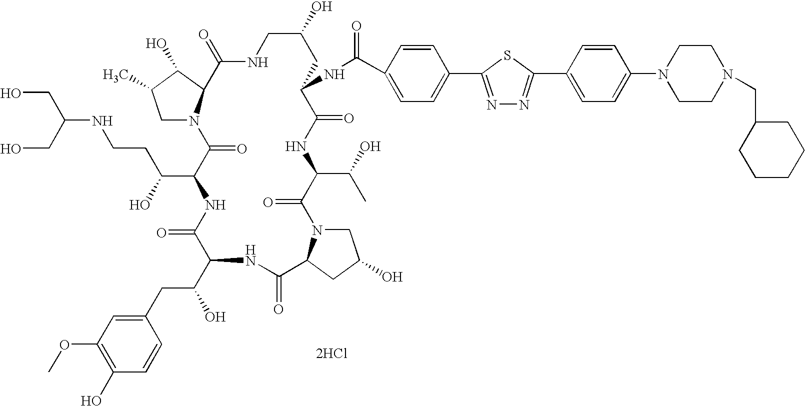 |
| 6 | 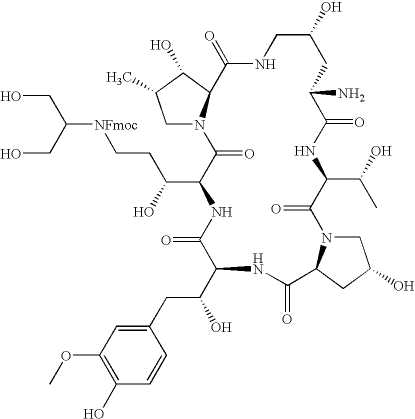 |
| | 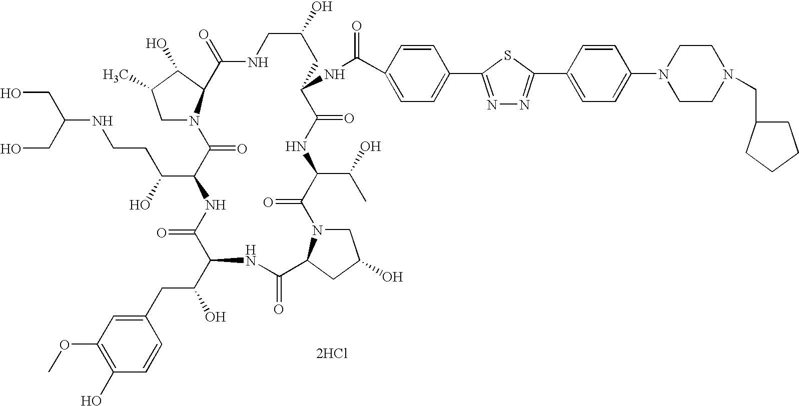 |
| 7 | 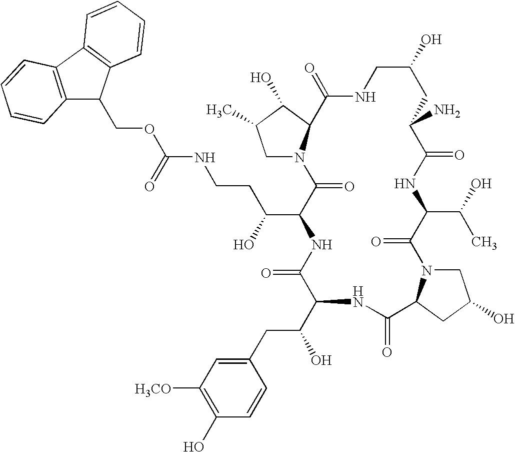 |
| | 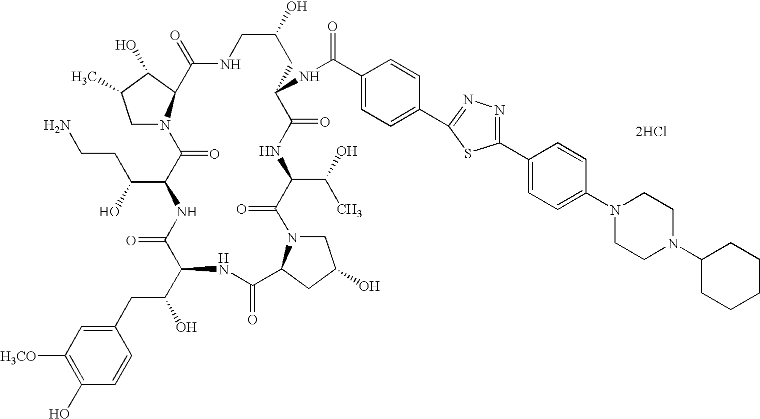 |
| 8 | 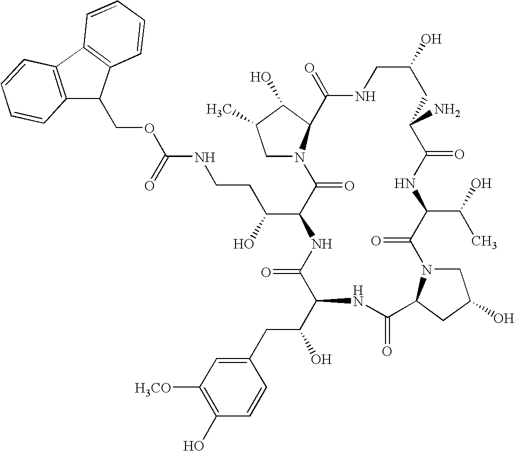 |
| | 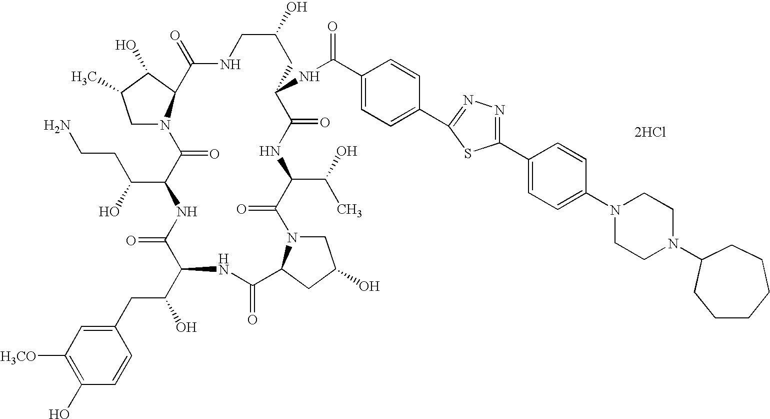 |
| 9 | 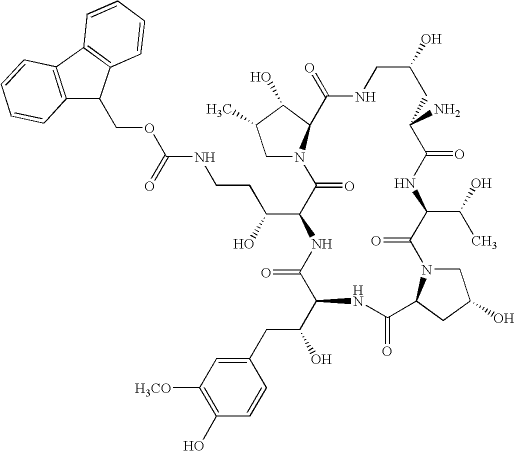 |
| | 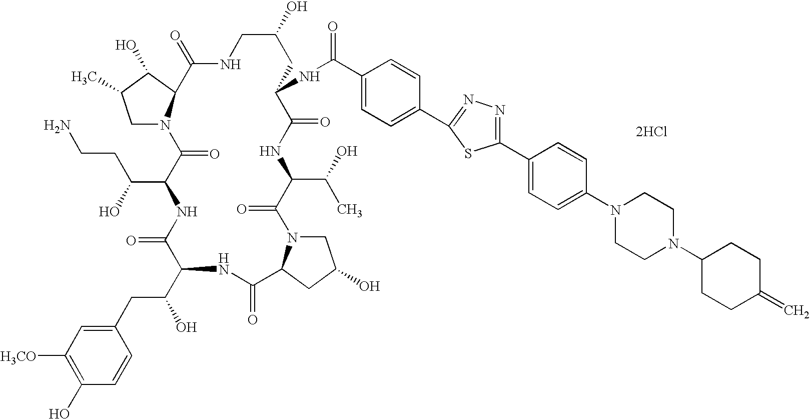 |
| 10 | 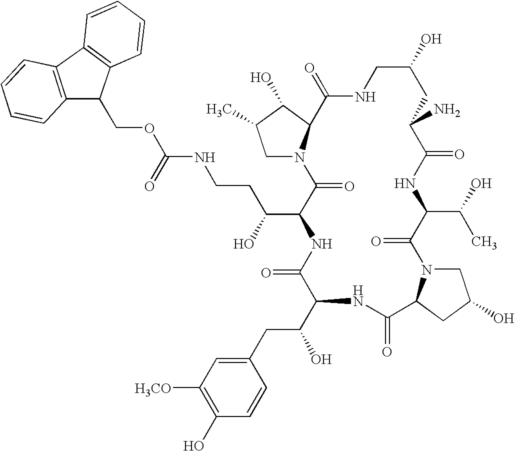 |
| | 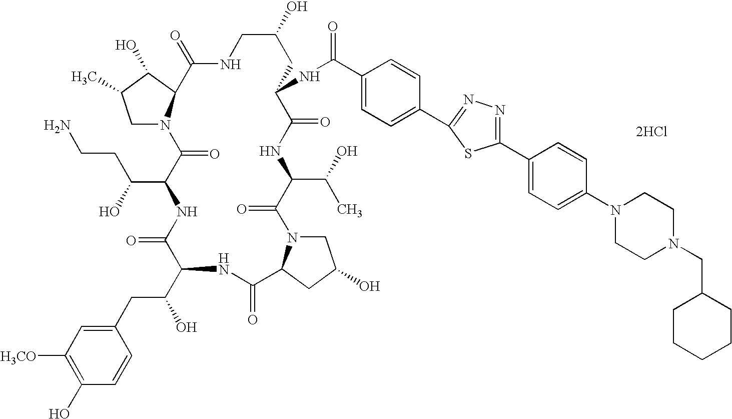 |
| 11 | 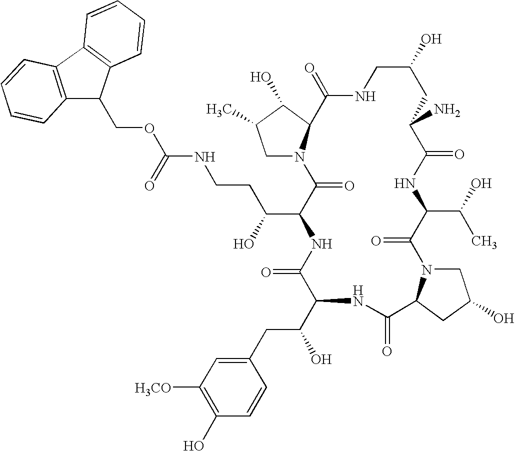 |
| | 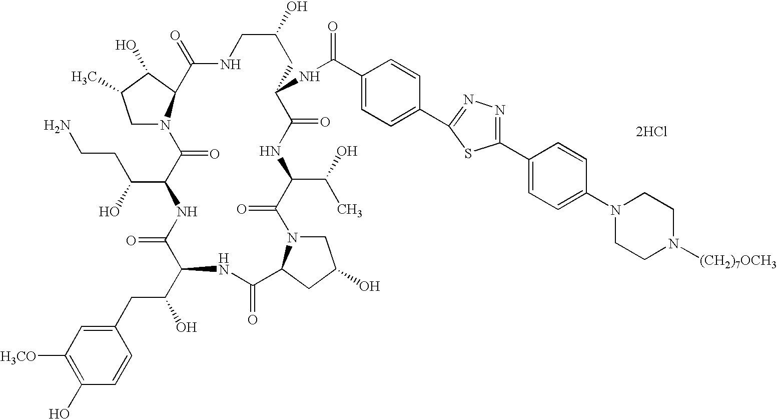 |
| 12 | 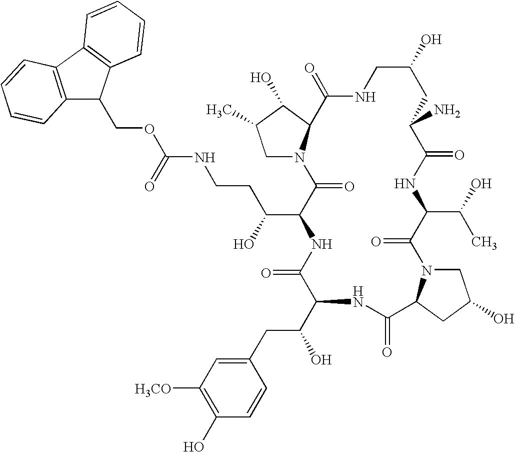 |
| | 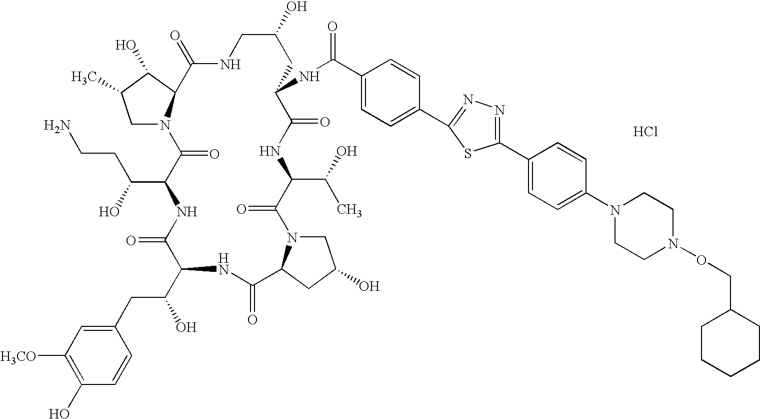 |
| 13 | 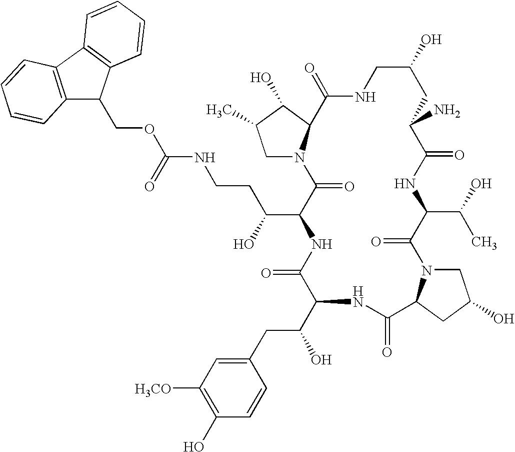 |
| | 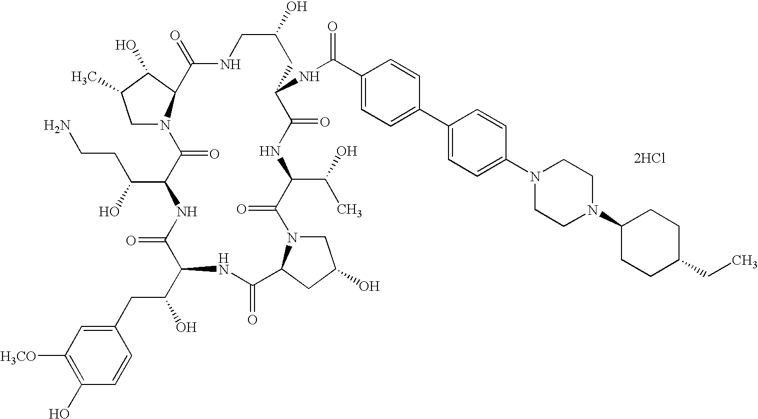 |
| 14 | 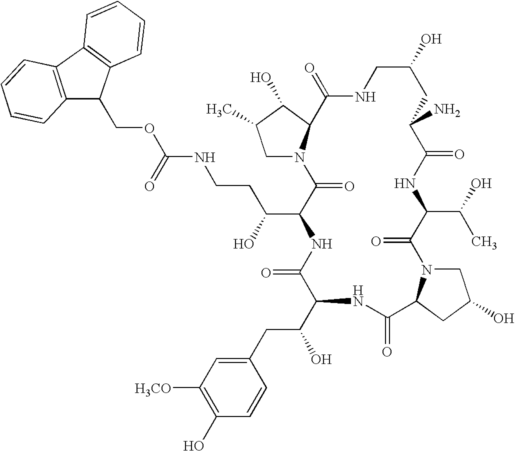 |
| | 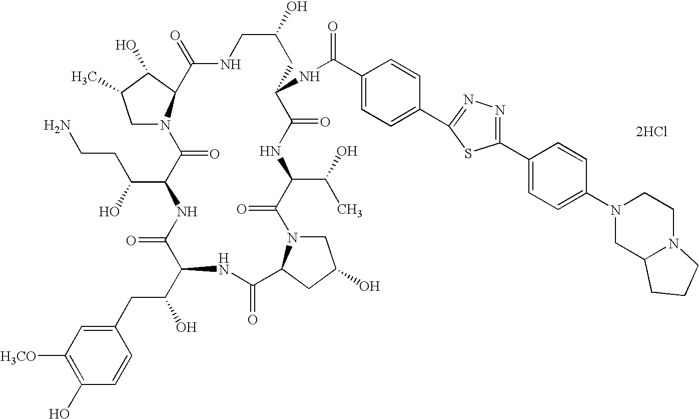 |
| 15 | 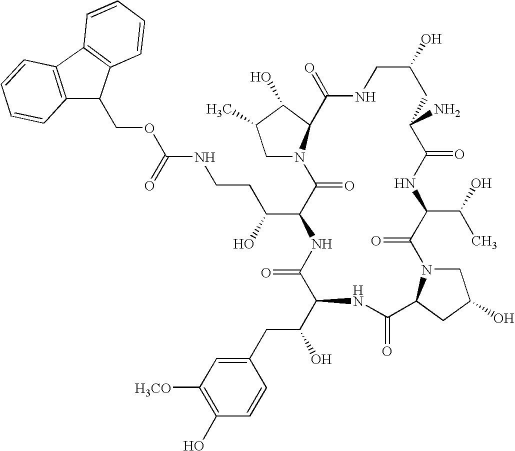 |
| | 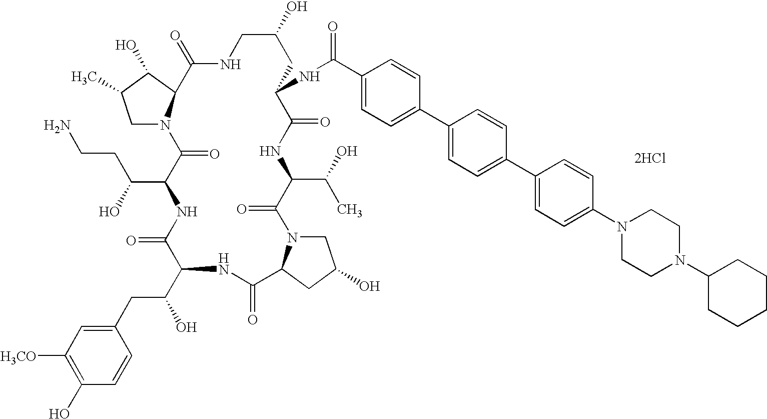 |
| 16 | 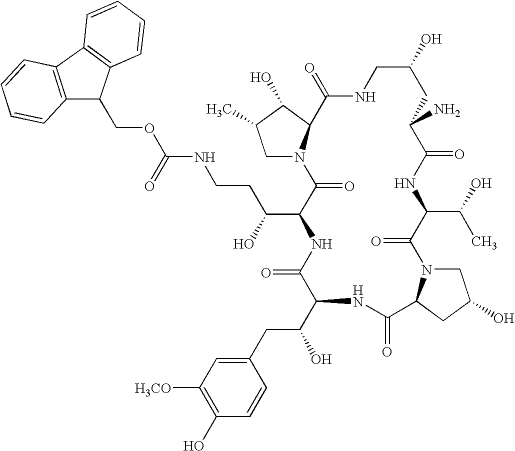 |
| | 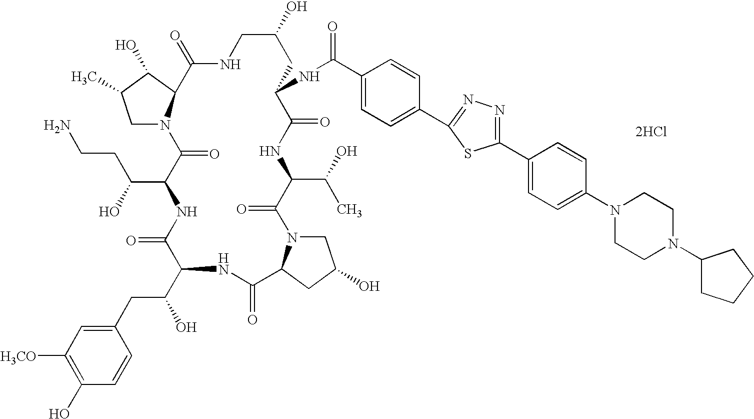 |
| 17 | 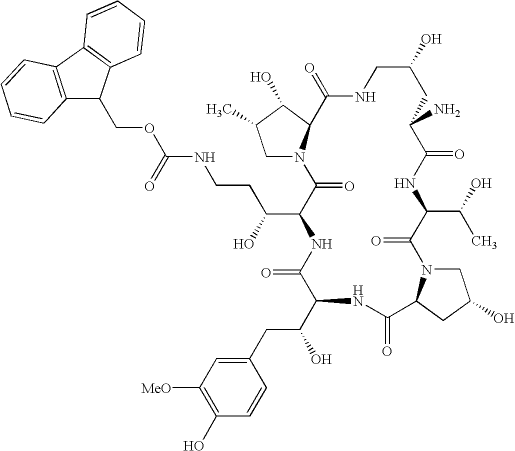 |
| | 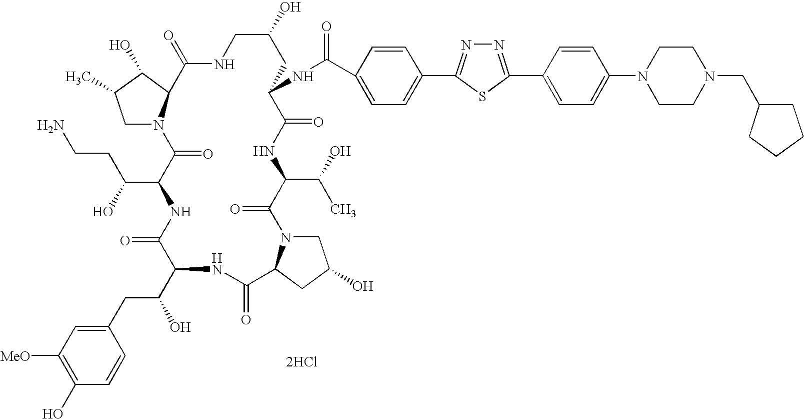 |
| 18 | 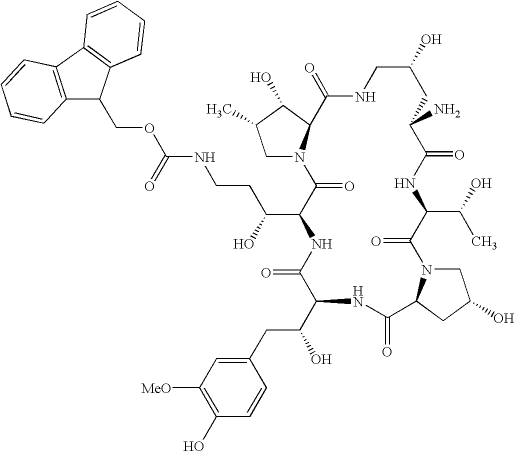 |
| | 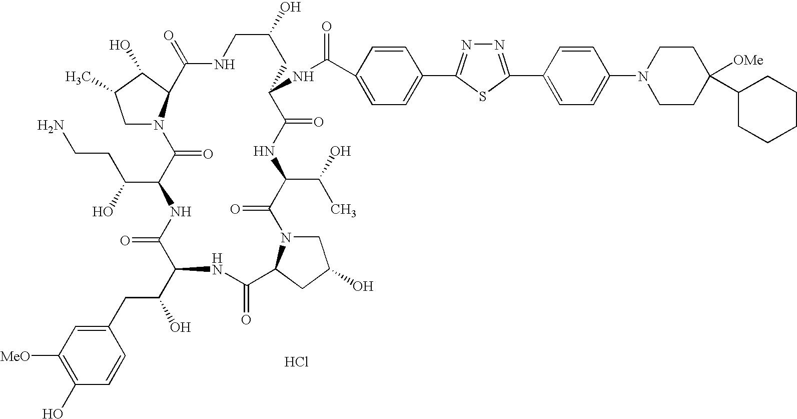 |
| 19 | 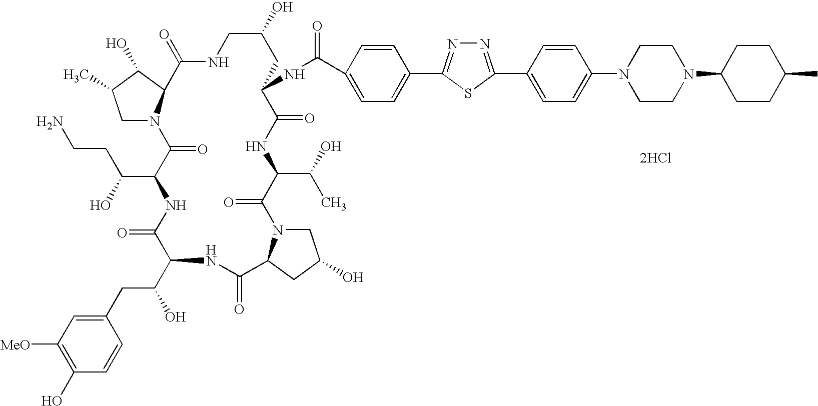 |
| | 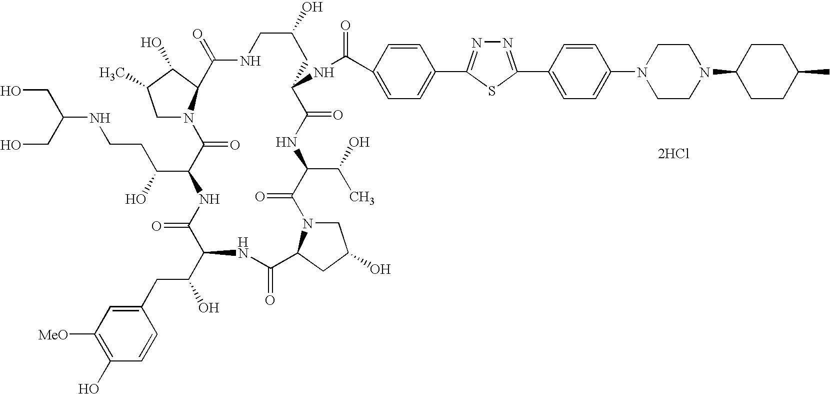 |
| 20 | 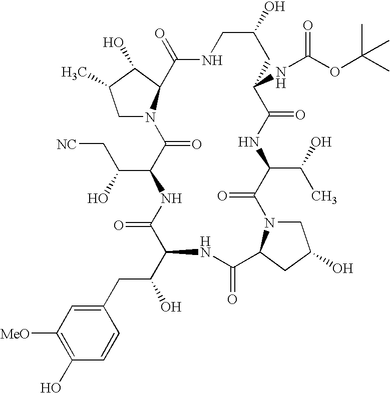 |
| | 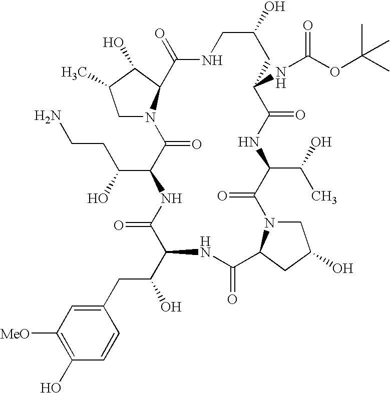 |
| 21 | 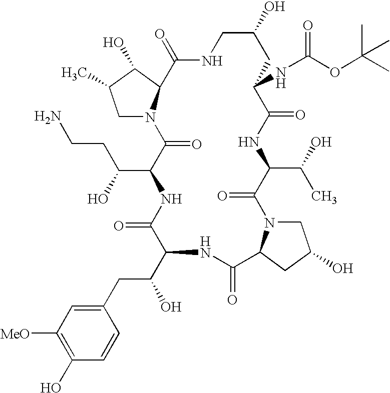 |
| | 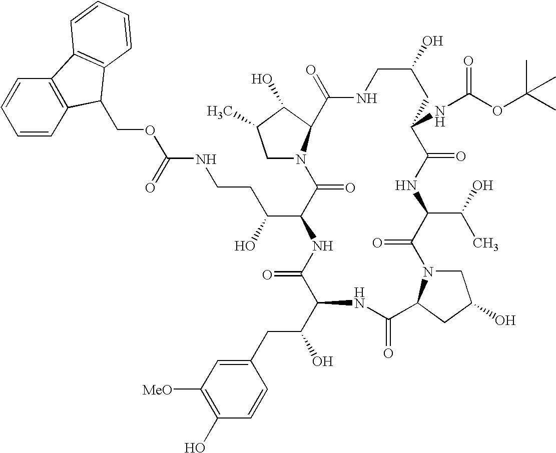 |
| 22 | 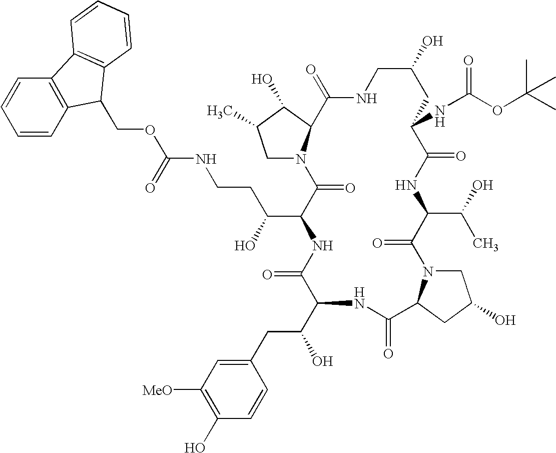 |
| | 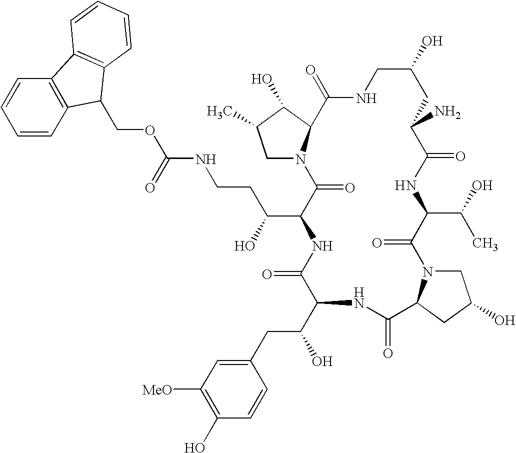 |
| 23 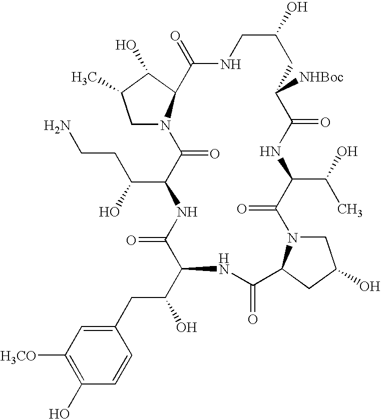 | |
| | 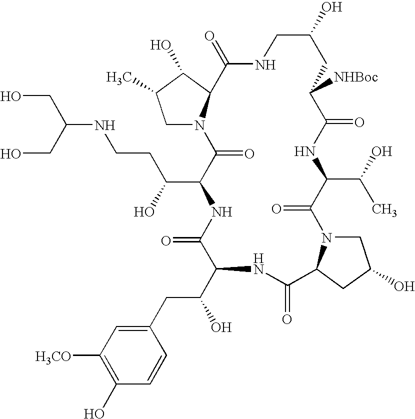 |
| 24 | 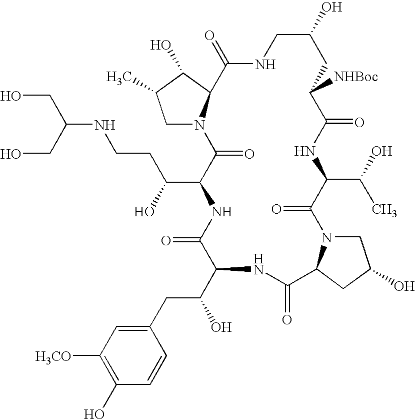 |
| | 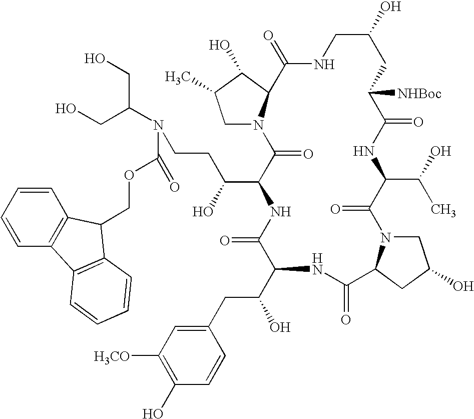 |
| 25 | 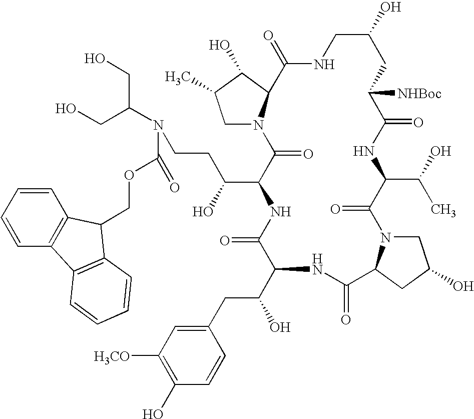 |
| | 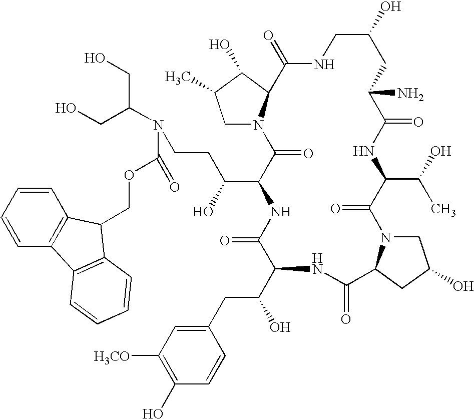 |

### EXAMPLE 1

To a mixture of starting compound (1) (10 g) and 1-[4-[5-[4-[4-(4-methylcyclohexyl)piperazinyl]phenyl]-1,3,4-thiadiazol-2-yl]benzoyloxy]-1H-1,2,3-benzotriazole (6.1 g) in N,N-dimethylformamide (100 ml) was added N,N-diisopropylethylamine (2.5 ml). After stirring for 6 hours at room temperature, to the reaction mixture was added piperidine (9.5 ml). After stirring for 40 hours at room temperature, the reaction mixture was poured into ethyl acetate. The resulting precipitate was collected and washed with ethyl acetate. The precipitate was dissolved in a mixture of water (2 L) and methanol (0.3 L) that was adjusted to pH 3 in advance and the solution was subjected to column chromatography on ODS (Daiso-gel, SP-120-40/60-ODS-B) (700 ml) eluting with 30%, 40%, 50% methanol in water that was adjusted to pH 3 in advance. The eluted fractions containing the object compound were collected and evaporated in vacuo. The residue was lyophilized to give object compound (1) (9.29 g).

IR (KBr): 1635, 1529, 1444, 1275, 1084 cm⁻¹

NMR (DMSO-d₆+D₂O, δ): 0.95 (3H, d, J=7.0 Hz), 0.97 (3H, d, J=7.0 Hz), 1.09 (3H, d, J=5.9 Hz), 1.4-2.7 (21H, m), 2.8-4.5 (24H, m), 3.74 (3H, s), 4.7-4.9 (2H, m), 6.5-6.7 (3H, m), 7.16 (2H, d, J=8.9 Hz), 7.92 (2H, d, J=8.8 Hz), 8.0-8.2 (4H, m)

MS (ESI+) m/z: 1291.4 (M+Na)

Elemental Analysis Calcd. for C₆₂H₈₆Cl₂N₁₂O₁₅S.7.5H₂O: C, 50.40; H, 6.89; N, 11.38. Found: C, 50.24; H, 6.95; N, 11.48.

The following compounds [Example 2 to 18] were obtained according to a similar manner to that of Example 1.

### EXAMPLE 2

NMR (DMSO-d₆+D₂O, δ): 0.8-1.3 (15H, m), 1.3-2.7 (15H, m), 2.8-4.3 (23H, m), 4.3-4.9 (8H, m), 6.5-6.75 (3H, m), 7.15 (1H, d, J=9.2 Hz), 7.95-8.3 (5H, m), 8.81 (1H, d, J=2.5 Hz)

MS (ESI+) m/z: 1284.27 (M+H)

### EXAMPLE 3

NMR (DMSO-d₆+D₂O, δ): 0.9-2.7 (17H, m), 0.97 (3H, d, J=6.7 Hz), 1.09 (3H, d, J=5.8 Hz), 2.75-4.55 (37H, m), 4.7-4.9 (2H, m), 6.5-6.75 (3H, m), 7.19 (2H, d, J=8.9 Hz), 7.85-8.2 (6H, m)

MS (ESI+) m/z: 1351.3 (M+Na)

### EXAMPLE 4

NMR (DMSO-d₆+D₂O, δ): 0.97 (3H, d, J=6.8 Hz), 1.08 (3H, d, J=5.5 Hz), 1.35-2.7 (19H, m), 2.75-4.55 (37H, m), 4.7-4.9 (2H, m), 6.5-6.75 (3H, m), 7.19 (2H, d, J=8.6 Hz), 7.8-8.15 (6H, m)

MS (ESI+) m/z: 1365.3 (M+Na)

### EXAMPLE 5

NMR (DMSO-d₆+D₂O, δ): 0.8-1.45 (5H, m), 0.97 (3H, d, J=6.8 Hz), 1.08 (3H, d, J=5.8 Hz), 1.55-2.7 (14H, m), 2.75-4.55 (37H, m), 4.7-4.9 (2H, m), 6.5-6.75 (3H, m), 7.19 (2H, d, J=8.8 Hz), 7.85-8.15 (6H, m)

MS (ESI+) m/z: 1365.3 (M+Na)

### EXAMPLE 6

NMR (DMSO-d₆+D₂O, δ): 0.85-4.55 (60H, m), 4.7-4.9 (2H, m), 6.5-6.75 (3H, m), 7.19 (2H, d, J=9.0 Hz), 7.94 (2H, d, J=9.0 Hz), 7.9-8.2 (4H, m)

MS (ESI+) m/z: 1351.4 (M+Na)

### EXAMPLE 7

IR (KBr): 3396.0, 1635.3, 1606.4, 1444.4 cm⁻¹ NMR (DMSO-d₆, δ): 0.96 (3H, d, J=6.8 Hz), 1.08 (3H, d, J=5.6 Hz), 1.19-5.50 (60H, m), 6.55-8.93 (16H, m)

MS (ESI+) m/z: 1277.4 (M+Na)

### EXAMPLE 8

IR (KBr): 3344.0, 1635.3, 1606.4, 1444.4 cm⁻¹

NMR (DMSO-d₆, δ): 0.96 (3H, d, J=6.7 Hz), 1.09 (3H, d, J=5.5 Hz), 1.23-5.51 (62H, m), 6.55-8.93 (16H, m)

MS (ESI+) m/z: 1269.4 (M+H), 1291.4 (M+Na)

### EXAMPLE 9

IR (KBr): 3434.6, 1658.5, 1635.3, 1546.6, 1444.4 cm⁻¹

NMR (DMSO-d₆, δ): 0.96 (3H, d, J=6.8 Hz), 1.09 (3H, d, J=5.6 Hz), 1.51-5.49 (60H, m), 6.55-8.93 (16H, m)

MS (ESI+) m/z: 1289.4 (M+Na)

### EXAMPLE 10

IR (KBr): 3396.0, 1633.4, 1608.3, 1521.6, 1444.4 cm⁻¹

NMR (DMSO-d₆, δ): 0.96 (3H, d, J=6.8 Hz), 1.09 (3H, d, J=5.9 Hz), 1.20-5.54 (62H, m), 6.55-8.92 (16H, m)

MS (ESI+) m/z: 1269.4 (M+H), 1291.4 (M+Na)

### EXAMPLE 11

MS (ESI+) m/z: 1301.5 (M+H), 1323.4 (M+Na)

### EXAMPLE 12

MS (ESI+) m/z: 1306.3 (M+Na)

### EXAMPLE 13

IR (KBr): 3342.0, 1633.4, 1519.6, 1496.5, 1446.4 cm⁻¹

NMR (DMSO-d₆, δ): 0.87 (3H, t, J=7.0 Hz), 0.96 (3H, d, J=6.7 Hz), 1.07 (3H, d, J=5.6 Hz), 1.15-5.50 (6H, m), 6.55-8.76 (16H, m)

MS (ESI+) m/z: 1199.5 (M+H), 1221.5 (M+Na)

### EXAMPLE 14

IR (KBr): 3367.1, 1633.4, 1602.6, 1517.7, 1444.4 cm⁻¹

NMR (DMSO-d₆, δ): 0.96 (3H, d, J=6.8 Hz), 1.08 (3H, d, J=5.6 Hz), 1.50-5.81 (51H, m), 6.55-8.92 (16H, m)

MS (ESI+) m/z: 1213.3 (M+H), 1235.3 (M+Na)

### EXAMPLE 15

IR (KBr): 3342.0, 1635.3, 1529.3, 1446.4 cm⁻¹

NMR (DMSO-d₆, 6): 0.89-5.52 (66H, m), 6.55-8.81 (20H, m)

MS (ESI+) m/z: 1247.3 (M+H), 1269.5 (M+Na)

### EXAMPLE 16

IR (KBr): 3336.2, 1633.4, 1602.6, 1444.4 cm⁻¹

NMR (DMSO-d₆, 6): 0.96 (3H, d, J=6.7 Hz), 1.08 (3H, d, J=5.6 Hz), 1.23-5.50 (58H, m), 6.56-8.92 (16H, m)

MS (ESI+) m/z: 1263.3 (M+Na)

### EXAMPLE 17

IR (KBr): 1645, 1632, 1537, 1524, 1450, 1421, 1273 cm⁻¹

NMR (DMSO-d₆+D₂O, δ): 0.97 (3H, d, J=6.8 Hz), 1.09 (3H, d, J=6.0 Hz), 1.2-2.7 (22H, m), 2.8-4.5 (24H, m), 3.74 (3H, s), 4.7-4.9 (2H, m), 6.5-6.7 (3H, m), 7.19 (2H, d, J=9.0 Hz), 7.94 (2H, d, J=8.7 Hz), 8.0-8.2 (4H, m)

MS (ESI+) m/z: 1277.3 (M+Na)

Elemental Analysis Calcd. for C₆₁H₈₄Cl₂N₁₂O₁₅S.9.5H₂O: C, 48.86; H, 6.92; N, 11.21. Found: C, 48.76; H, 7.01; N, 11.05.

### EXAMPLE 18

NMR (DMSO-d₆+D₂O, δ): 0.9-2.7 (26H, m), 0.97 (3H, d, J=6.8 Hz), 1.09 (3H, d, J=5.8 Hz), 2.8-4.5 (20H, m), 3.10 (3H, s), 3.75 (3H, s), 4.7-4.9 (2H, m), 6.5-6.8 (3H, m), 7.10 (2H, d, J=9.0 Hz), 7.86 (2H, d, J=8.8 Hz), 8.0-8.2 (4H, m)

MS (ESI+) m/z: 1306.3 (M+Na)

Elemental Analysis Calcd. for C₆₃H₈₆ClN₁₁O₁₆S.7H₂O: C, 52.29; H, 6.97; N, 10.65. Found: C, 52.09; H, 7.06; N, 10.56.

### EXAMPLE 19

To a mixture of starting compound (19) (0.5 g), dihydroxyacetone (67 mg) and acetic acid (64 μl) in N,N-dimethylformamide (3.5 ml) and methanol (7.5 ml) was added sodium cyanoborohydride (47 mg). After stirring for 72.5 hours at room temperature, the reaction mixture was poured into ethyl acetate. The resulting precipitate was collected and washed with ethyl acetate. The precipitate was dissolved in a mixture of water (300 ml) and methanol (30 ml) that was adjusted to pH 3 in advance and the solution was subjected to column chromatography on ODS (Daiso-gel, SP-120-40/60-ODS-B) (100 ml) eluting with 20% acetonitrile in water that was adjusted to pH 3 in advance. The eluted fractions containing the object compound were collected and evaporated in vacuo. The residue was lyophilized to give object compound (19) (0.50 g).

IR (KBr): 1649, 1632, 1539, 1524, 1450, 1421, 1275, 1084 cm⁻¹

NMR (DMSO-d₆+D₂O, δ): 0.97 (6H, d, J=7.1 Hz), 1.10 (3H, d, J=5.9 Hz), 1.5-2.7 (22H, m), 2.8-4.5 (28H, m), 3.75 (3H, s), 4.7-4.9 (0.2H, m), 6.5-6.7 (3H, m), 7.19 (2H, d, J=8.9 Hz), 7.94 (2H, d, J=8.7 Hz), 8.0-8.2 (4H, m)

MS (ESI+) m/z: 1365.6 (M+Na)

Elemental Analysis Calcd. for C₆₅H₉₂Cl₂N₁₂O₁₇S.9.5H₂O: C, 49.18; H, 7.05; N, 10.59. Found: C, 49.24; H, 7.00; N, 10.55.

### EXAMPLE 20

To a solution of starting compound (20) (35 g) in methanol (1 L) and water (0.2 L) was added cobalt(II) chloride hexahydrate (36 g). Then to the solution was added sodium borohydride (29 g) in small portions. After stirring for 4.5 hours at room temperature, the reacting mixture was poured into a mixture of methanol (1 L) and water (0.2 L). The solution was adjusted to pH 8 and filtrated. The filtrate was evaporated in vacuo and to the residue was added water (2 L). The solution was subjected to column chromatography on ODS (Daiso-gel, SP-120-40/60-ODS-B) (1 L) eluting with 20% acetonitrile in water. The eluted fractions containing the object compound were collected and evaporated in vacuo. The residue was lyophilized to give object compound (20) (29.7 g).

NMR (DMSO-d₆+D₂O, δ): 0.96 (3H, d, J=6.8 Hz), 1.08 (3H, d, J=5.7 Hz), 1.35 (9H, s), 1.2-3.0 (11H, m), 3.1-4.5 (16H, m), 3.74 (3H, s), 4.7-4.8 (2H, m), 6.5-6.7 (3H, m)

MS (ESI+) m/z: 925.3 (M+H)

The following compound was obtained according to a similar manner to that of Example 24.

### EXAMPLE 21

NMR (DMSO-d₆+D₂O, δ): 0.90 (3H, d, J=6.7 Hz), 1.09 (3H, d, J=5.3 Hz), 1.35 (9H, s), 1.2-2.7 (11H, m), 2.8-4.5 (19H, m), 3.74 (3H, s), 4.7-4.9 (2H, m), 6.5-6.7 (3H, m), 7.2-8.0 (8H, m)

MS (ESI+) m/z: 1169.3 (M+Na)

The following compound was obtained according to a similar manner to that of Example 25.

### EXAMPLE 22

NMR (DMSO-d₆+D₂O, δ): 0.8-4.5 (36H, m), 3.74 (3H, s), 4.7-4.9 (2H, m), 6.5-6.8 (3H, m), 7.2-8.0 (8H, m)

MS (ESI+) m/z: 1069.3 (M+Na)

### EXAMPLE 23

To a mixture of starting compound (23)(40 g), dihydroxyacetone (8.57 g) and acetic acid (6.68 ml) in methanol (400 ml) was added sodium cyanoborohydride (5.98 g). After stirring for 16.5 hours at room temperature, the reaction mixture was poured into ethyl acetate. The resulting precipitate was collected and washed with ethyl acetate. The precipitate was dissolved with water (10 L), and the solution was subjected to column chromatography on ODS (Daiso-gel,SP-120-40/60-ODS-B) (1100 mL) eluting with 20% acetonitrile in water. The eluted fractions containing the object compound were collected and evaporated in vacuo. The residue was lyophilized to give object compound (23)(31.51 g).

IR (KBr): 3297.7, 1660.4, 1631.5, 1519.6 cm⁻¹

NMR (DMSO-d₆, δ): 0.95 (3H, d, J=6.7 Hz), 1.10 (3H, d, J=5.4 Hz), 1.35 (9H, s), 1.55-5.20 (47H, m), 6.53-7.93 (8H, m)

MS (ESI+) m/z: 999.4 (M+H), 1021.3 (M+Na)

### EXAMPLE 24

To a solution of starting compound (24) (31 g) and N,N-diisopropylethylamine (13.5 ml) in N,N-dimethylformamide (217 ml) was added 9-fluorenylmethyl chloroformate (12 g) at ambient temperature, and the mixture was stirred for 1.5 hour. The reaction mixture was dissolved in a mixture of 30% acetonitrile in water (4500 ml), and the solution was subjected to column chromatography on ODS (Daiso-gel, SP-120-40/60-ODS-B (Trademark: prepared by Daiso Co., Ltd.)) (1100 ml) eluting with 40% acetonitrile in water. The fractions containing the object compound were collected and evaporated under reduced pressure to remove acetonitrile. The residue was lyophilized to give object compound (24) (17.54 g).

IR (KBr): 3345.9, 1666.2, 1633.4, 1517.7, 1448.3 cm⁻¹

NMR (DMSO-d₆, δ): 0.87-0.96 (3H, m), 1.03-1.12 (3H, m), 1.35 (9H, s), 1.57-5.21 (49H, m), 6.52-7.99 (16H, m)

MS (ESI+) m/z: 1243.3 (M+Na)

### EXAMPLE 25

To a suspension of starting compound (25) (17.5 g) and triethylsilane (18.3 ml) in dichloromethane (263 ml) was added dropwise trifluoroacetic acid (33.1 ml) with stirring at ambient temperature, and the mixture was stirred at ambient temperature for 2.5 hours. The reaction mixture was poured into diisopropylethyl ether (2400 ml). The resulting precipitate was collected by filtration, and the precipitate was dissolved with 20% acetonitrile in water, and adjusted to pH 7.8 with saturated sodium hydrogen carbonate in water. The solution was subjected to column chromatography on ODS (Daiso-gel, SP-120-40/60-ODS-B (Trademark: prepared by Daiso Co., Ltd.)) (700 ml) eluting with 25%-35% acetonitrile in water. The fractions containing the object compound were collected and evaporated under reduced pressure to remove acetonitrile. The residue was lyophilized to give object compound (25) (13.38 g).

IR (KBr): 3376.7, 1660.4, 1633.4, 1517.7, 1446.4 cm⁻¹

NMR (DMSO-d₆, δ): 0.85-0.91 (3H, m), 1.08 (3H, d, J=5.7 Hz), 1.50-5.21 (51H, m), 6.52-8.00 (15H, m)

MS (ESI+) m/z: 1121.3 (M+H), 1143.4 (M+Na)

Abbreviations and Acronyms

| Abbreviations | |
|---|---|
| and Acronyms | Full Name |
| AcOEt or EtOAc | ethyl acetate |
| AcOH | acetic acid |
| BuOH, t-BuOH, | butanol, t-butyl alcohol, etc. |
| etc. | |
| DME | 1,2-dimethoxyethane |
| DMF | N,N-dimethylformamide |
| DMSO | dimethyl sulfoxide |
| Et3N | triethylamine |
| EtOH | ethanol |
| IPE | diisopropyl ether |
| MeOH | methanol |
| PrOH, i-PrOH, | propanol, isopropyl alcohol, etc. |
| etc. | |
| TFA | trifluoroacetic acid |
| THF | tetrahydrofuran |
| EDCI | 1-ethyl-3-[3′-(dimethylamino)propyl] carbodiimide |
| HOBt or HOBT | 1-hydroxybenztriazole |
| BSU | bis(trimethylsilyl) urea |
| MSA | mono (trimethylsilyl) acetamide |
| Pd/C | palladium on carbon |
| deg | ° C. = degree centigrade |
| min | minute(s) |
| hr or h | hour(s) |
| conc. | concentrated |
| aq | aqueous (ex. ag NaHCO3 solution) |
| Boc | Tert-butoxycarbonyl |
| Fmoc | fuluorenylmethoxycarbonyl |

1. A lipopeptide compound of the following general formula (I): 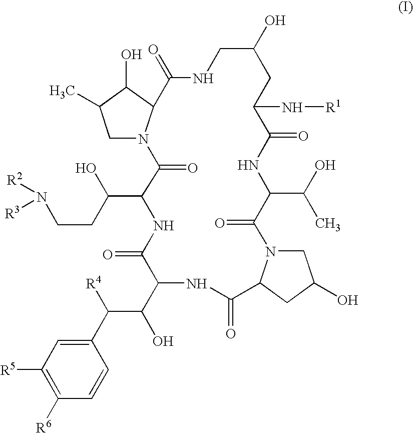
wherein
R¹ is hydrogen or acyl group,
R² is hydrogen or amino protective group,
R³ is hydrogen or lower alkyl substituted with one or more hydroxy,
R⁴ is hydrogen or hydroxy,
R⁵ is lower alkoxy, and
R⁶ is hydroxy or acyloxy,
or a salt thereof.

2. A compound of claim 1, wherein
R¹ is hydrogen, lower alkoxycarbonyl or benzoyl substituted with one or more suitable substituent(s),
R² is hydrogen,
R³ is hydrogen or lower alkyl substituted with two hydroxy,
R⁴ is hydrogen,
R⁵ is lower alkoxy, and
R⁶ is hydroxy.

3. A compound of claim 2, wherein
R¹ is benzoyl substituted with a suitable substituent, and
R⁵ is methoxy.

4. A compound of claim 3, wherein
R¹ is
1) benzoyl substituted with heterocyclic group substituted with phenyl substituted with optionally substituted heterocyclic group,
2) benzoyl substituted with heterocyclic group substituted with heterocyclic group substituted with optionally substituted heterocyclic group,
3) benzoyl substituted with phenyl substituted with optionally substituted heterocyclic group,
4) benzoyl substituted with phenyl substituted with phenyl substituted with optionally substituted heterocyclic group,
R² is hydrogen,
R³ is hydrogen or lower alkyl substituted with two hydroxy,
R⁴ is hydrogen,
R⁵ is methoxy, and
R⁶ is hydroxy.

5. A compound of claim 4, wherein
R¹ is
1) benzoyl substituted with thiadiazolyl substituted with phenyl substituted with piperazinyl substituted with cyclo(lower)alkyl,
2) benzoyl substituted with thiadiazolyl substituted with phenyl substituted with piperazinyl substituted with cyclo(higher)alkyl,
3) benzoyl substituted with thiadiazolyl substituted with phenyl substituted with piperazinyl substituted with cyclo(lower)alkyl substituted with lower alkylidene,
4) benzoyl substituted with thiadiazolyl substituted with phenyl substituted with piperazinyl substituted with cyclo(lower)alkyl(lower)alkyl,
5) benzoyl substituted with thiadiazolyl substituted with phenyl substituted with piperazinyl substituted with lower alkoxy(higher)alkyl,
6) benzoyl substituted with thiadiazolyl substituted with phenyl substituted with piperidyl substituted with cyclo(lower)alkyl(lower)alkoxy,
7) benzoyl substituted with phenyl substituted with piperazinyl substituted with cyclo(lower)alkyl substituted with lower alkyl,
8) benzoyl substituted with thiadiazolyl substituted with phenyl substituted with hexahydropyrrolopyrazinyl,
9) benzoyl substituted with phenyl substituted with phenyl substituted with piperazinyl substituted with cyclo(lower)alkyl,
10) benzoyl substituted with thiadiazolyl substituted with phenyl substituted with piperazinyl substituted with cyclo(lower)alkyl substituted with lower alkyl,
11) benzoyl substituted with thiadiazolyl substituted with pyridyl substituted with piperazinyl substituted with cyclo(lower)alkyl substituted with lower alkyl,
12) benzoyl substituted with thiadiazolyl substituted with phenyl substituted with piperidyl substituted with lower alkoxy and cyclo(lower)alkyl,
R² is hydrogen,
R³ is hydrogen or lower alkyl substituted with two hydroxy,
R⁴ is hydrogen,
R⁵ is methoxy, and
R⁶ is hydroxy.

6. A compound of claim 5, wherein
R¹ is benzoyl substituted with thiadiazolyl substituted with phenyl substituted with piperazinyl substituted with cyclo(lower)alkyl,
R² is hydrogen,
R³ is hydrogen or lower alkyl substituted with two hydroxy,
R⁴ is hydrogen,
R⁵ is methoxy, and
R⁶ is hydroxy.

7. A process for preparing a lipopeptide compound (I) of claim 1, or a salt thereof,
which comprises,
i) reducing a compound (II) of the formula: 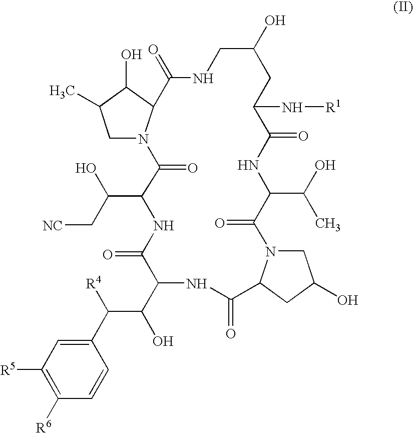
wherein R¹, R⁴, R⁵ and R⁶ are defined in claim 1, or a salt thereof to give a compound (Ia) of the formula: 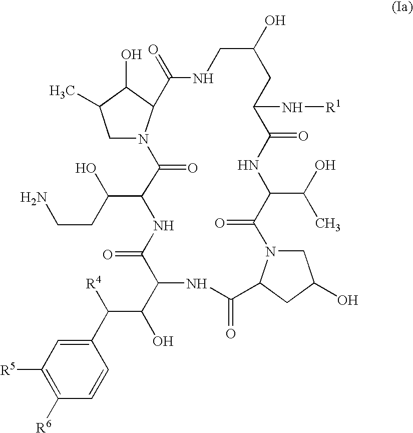
wherein R¹, R⁴, R⁵ and R⁶ are defined above, or a salt thereof, or
ii) reacting a compound (Ia) of the formula: 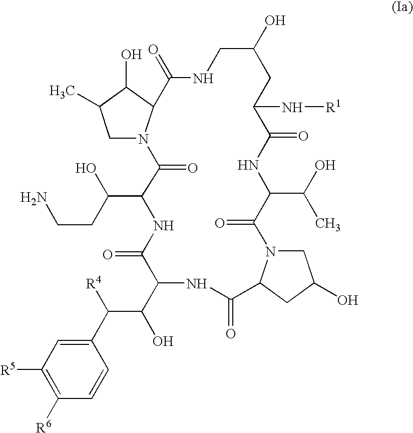
wherein R¹, R⁴, R⁵ and R⁶ are defined in claim 1, or its reactive derivative at the amino group or a salt thereof, with a compound (III) of the formula:
Rₐ³═O (III)
wherein Rₐ³ is lower alkyl substituted with one or more hydroxy,
or its reactive derivative or a salt thereof, to give a compound (Ib) of the formula: 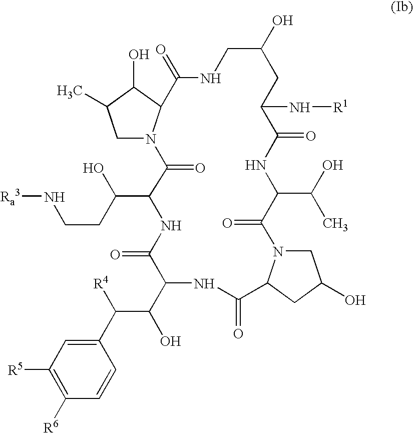
wherein R¹, Rₐ³, R⁴, R⁵ and R⁶ are defined above, or a salt thereof, or
iii) subjecting a compound (Ic) of the formula: 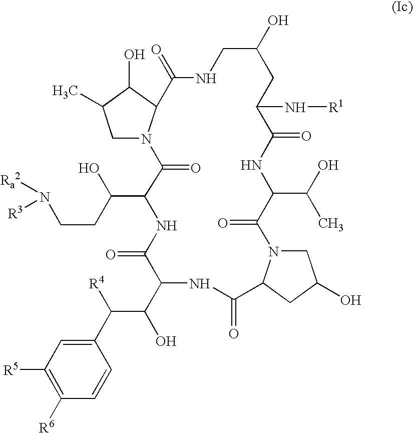
wherein R¹, R³, R⁴, R⁵ and R⁶ are defined in claim 1, Rₐ² is amino protective group,
or a salt thereof, to elimination reaction of the amino protective group, to give a compound (Id) of the formula: 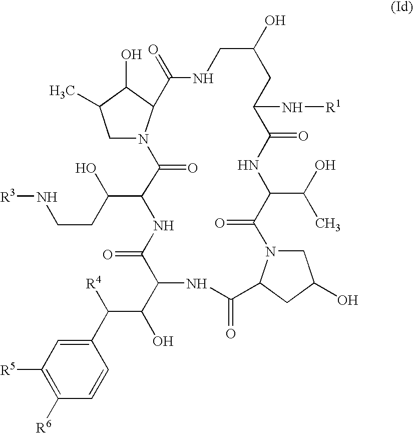
wherein R¹, R³, R⁴, R⁵ and R⁶ are defined above, or a salt thereof, or
iv) reacting a compound (Ie) of the formula: 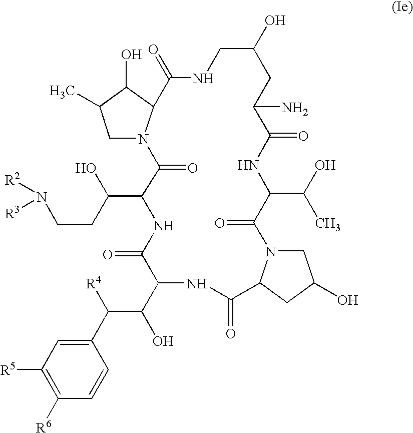
wherein R², R³, R⁴, R⁵ and R⁶ are defined in claim 1, or its reactive derivative at the amino group or a salt thereof, with a compound (IV) of the formula:
Rₐ¹—OH (IV)
wherein Rₐ¹ is acyl group,
or its reactive derivative or a salt thereof, to give a compound (If) of the formula: 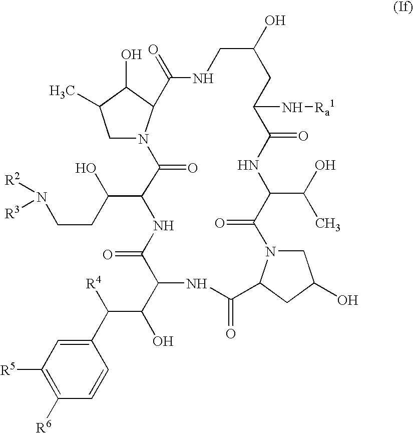
wherein R², R³, R⁴, R⁵ and R⁶ are defined in claim 1, Rₐ¹ is defined above, or a salt thereof.

8. A pharmaceutical composition which comprises, as an active ingredient, a compound of claim 1 or a pharmaceutically acceptable salt thereof in admixture with pharmaceutically acceptable carriers or excipients.

9. Use of a compound of claim 1 or a pharmaceutically acceptable salt thereof for the manufacture of a medicament.

10. A compound of claim 1 or a pharmaceutically acceptable salt thereof for use as a medicament.

11. A method for the prophylactic and/or therapeutic treatment of infectious diseases caused by pathogenic microorganisms, which comprises administering a compound of claim 1 or a pharmaceutically acceptable salt thereof to a human being or an animal.

12. A commercial package comprising the pharmaceutical composition of claim 8 and a written matter associated therewith, wherein the written matter states that the pharmaceutical composition can or should be used for preventing or treating infections disease.

13. An article of manufacture, comprising packaging material and the compound (I) identified in claim 1 contained within said packaging material, wherein said the compound (I) is therapeutically effective for preventing or treating infectious diseases, and wherein said packaging material comprises a label or a written material which indicates that said compound (I) can or should be used for preventing or treating infectious diseases.
